(19) United States
(12) Patent Application Publication
(10) Pub. No.: US 2025/0319071 A1
(43) Pub. Date: Oct. 16, 2025

(54) PHARMACEUTICAL COMPOSITION COMPRISING TEGOPRAZAN AND NON-STEROIDAL ANTI-INFLAMMATORY DRUGS

(71) Applicant: HK INNO.N CORPORATION, Cheongju-si Chungcheongbuk-do (KR)

(72) Inventors: Min Jung KIM, Seoul (KR); Young Dae CHO, Gyeonggi-do (KR); Joo-hwan KIM, Gyeonggi-do (KR); Da Som LIM, Gyeonggi-do (KR); Myeongjoong KIM, Gyeonggi-do (KR); Sun Young Park, Gyeonggi-do (KR); Eun Kyung JEON, Gyeonggi-do (KR); Tae Keun CHO, Incheon (KR)

(21) Appl. No.: 18/269,421

(22) PCT Filed: Dec. 29, 2021
(86) PCT No.: PCT/IB2021/062408

§ 371 Date: Jun. 23, 2023

(30) Foreign Application Priority Data
Dec. 30, 2020 (KR) 10-2020-0188540

(51) Int. Cl. A61K 31/4184; A61K 9/24; A61K 31/192

(52) U.S. Cl. CPC A61K 31/4184; A61K 9/209; A61K 31/192

(57) ABSTRACT

The present invention relates to a pharmaceutical composition including non-steroidal anti-inflammatory drugs (NSAIDs) and tegoprazan as an active ingredient.

### TECHNICAL FIELD

The present invention relates to a pharmaceutical composition including tegoprazan and non-steroidal anti-inflammatory drugs and a method for preparing the same.

### BACKGROUND

Non-steroidal anti-inflammatory drugs (NSAIDs) are drugs with analgesic, antipyretic, and anti-inflammatory actions and thus are used in the treatment of acute or chronic diseases with pain and inflammation, such as migraines, arthralgia, myalgia, rheumatoid arthritis, gout or the like.

However, with the aging of a drug-using population, the use of NSAIDs increases due to the rise in musculoskeletal and cardiovascular diseases, and thus side effects caused by the administration thereof are also becoming a problem. In particular, it is known that there is a risk of developing NSAID-associated ulcers among patients in need of continuous NSAIDs treatment for chronic diseases or disorders (pain and inflammation). The side effects that occur in 20-40% of NSAIDs users are mostly found in the stomach and small intestine, and are typically involved in gastrointestinal diseases showing symptoms of indigestion (gastric pain, heartburn, bloat or nausea), erosion, gastritis/duodenitis, ulcer and the like, as well as anemia due to excessive bleeding. Even if side effects such as gastrointestinal diseases, bleeding or the like are treated, patients who need continuous NSAIDs treatment for chronic diseases or disorders (pain and inflammation) continue to take NSAIDs, and thus frequently develop the recurrence of side effects such as gastrointestinal diseases, bleeding or the like.

Accordingly, there is a need for research and development for controlling side effects caused by non-steroidal anti-inflammatory drugs.

### DISCLOSURE

### Technical Problem

An object of the present invention is to provide a pharmaceutical composition including tegoprazan and non-steroidal anti-inflammatory drugs.

An object of the present invention is to provide a method for preparing a pharmaceutical composition including tegoprazan and non-steroidal anti-inflammatory drugs.

An object of the present invention is to provide a method for preventing or treating pain diseases, inflammatory diseases and/or gastrointestinal diseases, including administering a pharmaceutical composition containing tegoprazan and non-steroidal anti-inflammatory drugs.

An object of the present invention is to provide a use of a pharmaceutical composition including tegoprazan and non-steroidal anti-inflammatory drugs for preventing or treating pain diseases, inflammatory diseases and/or gastrointestinal diseases.

An object of the present invention is to provide a use of a pharmaceutical composition including tegoprazan and non-steroidal anti-inflammatory drugs in preparation of a drug for preventing or treating pain diseases, inflammatory diseases and/or gastrointestinal diseases.

### Technical Solution

Each description and embodiment disclosed in the present invention may also be applied to other descriptions and embodiments thereof, respectively. In other words, all the combinations of various elements disclosed in the present invention fall within the scope of the present invention. In addition, it may not be seen that the scope of the present invention is limited to the specific descriptions described below.

Furthermore, in the present specification, the first and second, the upper and lower, etc. are used only for classification, and may not be regarded to specify any order or position.

In addition, in the present specification, singular expressions include plural expressions, and plural expressions include singular expressions, unless specified otherwise in the context thereof.

The present invention may provide a pharmaceutical combination preparation (complex) in which tegoprazan may be administered simultaneously with non-steroidal anti-inflammatory drugs (NSAIDs).

The present invention may provide a pharmaceutical composition designed to have an immediate release region (compartment) and an enteric region (compartment) separated from each other with improved stability while minimizing gastrointestinal side effects caused by the use of non-steroidal drugs, and a method for preparing the same.

The present invention may provide:
(1) A pharmaceutical composition comprising:
a first compartment containing a first active ingredient; and a second compartment containing a second active ingredient,
wherein the first active ingredient is subjected to controlled release, and the second active ingredient is subjected to immediate release,
in which the first active ingredient is non-steroidal anti-inflammatory drug (NSAID), and
the second active ingredient is tegoprazan, an optical isomer thereof, a pharmaceutically acceptable salt thereof, a hydrate or solvate thereof, or a mixture thereof.
(2) The pharmaceutical composition of (1), wherein the tegoprazan or the pharmaceutically acceptable salt thereof is in an amorphous or crystalline form.
(3) The pharmaceutical composition of any one of (1) to (2), wherein the pharmaceutical composition is a unit dosage form of a combination preparation comprising both the first active ingredient and the second active ingredient.
(4) The pharmaceutical composition of any one of (1) to (3), wherein the unit dosage form comprises a first compartment containing a first active ingredient, and a second compartment containing a second active ingredient,
in which the first compartment containing the first active ingredient includes particle containing:
a core having the first active ingredient; and an enteric coated layer located on the core and surrounded the core.
(5) The pharmaceutical composition of any one of (1) to (4), wherein the particle is tablet, pellet or granule.
(6) The pharmaceutical composition of any one of (1) to (5), wherein the unit dosage form is tablet.
(7) The pharmaceutical composition of any one of (1) to (6), wherein the second compartment comprising the second active ingredient is a second active ingredient layer containing the second active ingredient.
(8) The pharmaceutical composition of any one of (1) to (7), wherein the second active ingredient layer is a coated layer located on and surrounded the first compartment comprising the first active ingredient.
(9) The pharmaceutical composition of any one of (1) to (8), wherein the second active ingredient layer is laminated on the first compartment comprising the first active ingredient.
(10) The pharmaceutical composition of any one of (1) to (9), wherein the second active ingredient layer comprises particle containing the second active ingredient.
(11) The pharmaceutical composition of any one of (1) to (10), wherein the particle comprising the second active ingredient is tablet, pellet or granule.
(12) The pharmaceutical composition of any one of (1) to (11), wherein a separation layer for blocking a contact between the first active ingredient and the second active ingredient is further comprised at least either: between the core and the enteric coated layer of the first compartment; and between the first compartment and the second compartment.
(13) The pharmaceutical composition of any one of (1) to (12), wherein the unit dosage form is a capsule.
(14) The pharmaceutical composition of any one of (1) to (13), wherein the first compartment comprising the first active ingredient is particle comprising:
a core containing the first active ingredient; and
an enteric coated layer located on the core and surrounded the core,
and wherein the second compartment comprising the second active ingredient is particle comprising the second active ingredient.
(15) The pharmaceutical composition of any one of (1) to (14), wherein the capsule is filled with particle comprising the first active ingredient and particle comprising the second active ingredient.
(16) The pharmaceutical composition of any one of (1) to (15), wherein the particle comprising the first active ingredient and the particle comprising the second active ingredient are each independently tablet, pellet, granule, or mixture thereof.
(17) The pharmaceutical composition of any one of (1) to (16), wherein the capsule is filled with tablet,
in which the tablet comprises the first compartment containing the first active ingredient; and the second compartment containing the second active ingredient, in which the first compartment containing the first active ingredient includes a particle containing:
a core having the first active ingredient; and
an enteric coated layer located on the core and surrounded the core, and
the second compartment containing the second active ingredient includes a particle containing the second active ingredient or a second active ingredient layer containing the second active ingredient.
(18) The pharmaceutical composition of any one of (1) to (17), wherein the enteric coated layer is about 5 wt % or more and about 20 wt % or less based on the total weight of the core.
(19) The pharmaceutical composition of any one of (1) to (18), wherein the non-steroidal anti-inflammatory drug comprises naproxen or a pharmaceutically acceptable salt thereof.
(20) The pharmaceutical composition of any one of (1) to (19), wherein the pharmaceutical composition is a composition for preventing or treating inflammation and/or pain diseases.
(21) The pharmaceutical composition of any one of (1) to (20), wherein the pharmaceutical composition is a composition for preventing or treating inflammation and/or pain diseases and gastrointestinal diseases.
(22) The pharmaceutical composition of any one of (1) to (21), wherein the gastrointestinal diseases are caused by non-steroidal anti-inflammatory drugs.
(23) A method for preventing or treating pain diseases, inflammatory diseases and/or gastrointestinal diseases, comprising administering the pharmaceutical composition of any one of (1) to (22) to a subject in need thereof.
(24) A use of the pharmaceutical composition of any one of (1) to (22) for preventing or treating pain diseases, inflammatory diseases and/or gastrointestinal diseases.
(25) A use of the pharmaceutical composition of any one of (1) to (22) in preparation of a drug for preventing or treating pain diseases, inflammatory diseases and/or gastrointestinal diseases.

The present invention may provide a pharmaceutical composition which includes:
a first compartment containing a first active ingredient; and a second compartment containing a second active ingredient,
in which the first active ingredient is subjected to controlled release, and the second active ingredient is subjected to immediate release,
in which the first active ingredient is non-steroidal anti-inflammatory drug (NSAID), and
the second active ingredient is tegoprazan, an optical isomer thereof, a pharmaceutically acceptable salt thereof, a hydrate or solvate thereof, or a mixture thereof.

Non-steroidal anti-inflammatory drugs (NSAIDs) are drugs with anti-inflammatory, analgesic and antipyretic actions and thus are used for treating a wide variety of acute or chronic pain diseases and inflammatory diseases such as migraines, arthralgia, myalgia, rheumatoid arthritis, gout or the like. In the present invention, the non-steroidal anti-inflammatory drugs may be referred to interchangeably with non-steroidal antiphlogistic drugs, non-steroidal anti-inflammatory drugs, or NSAID(s).

In the present invention, the NSAID may be selected from the group consisting of aspirin (acetyl salicylic acid), diclofenac, aceclofenac, etodolac, indometacin, nabumetone, sulindac, benoxaprofen, carprofen, dexibuprofen, dexketoprofen, fenbufen, fenoprofen, flunoxaprofen, flurbiprofen, ketoprofen, ibuprofen, ketorolac, loxoprofen, miroprofen, naproxen, oxaprozin, pirprofen, mefenamic acid, flufenamic acid, meclofenamic acid, piroxicam, droxicam, lornoxicam, meloxicam, tenoxicam, celecoxib, deracoxib, etoricoxib, firocoxib, lumiracoxib, parecoxib, rofecoxib, valdecoxib, nimesulide, ketorolac, azapropazone, tolfenamic acid, sulindac, diflunisal, tiaprofenic acid, podophyllotoxin derivatives, acemetacin, oxaprozin, floctafenine, phenylbutazone, proglumetacin, flurbiprofen, tolmethine, fenbufen, pharmaceutically acceptable salts thereof, precursors thereof and mixtures thereof. Preferably, the NSAID may be naproxen or a pharmaceutically acceptable salt thereof, but is not necessarily limited thereto.

In embodiments of the present invention, the pharmaceutical composition may contain the NSAID in an amount of about 7.5 mg to about 2000 mg, preferably about 100 mg to about 1500 mg, and more preferably about 250 mg to about 1000 mg per unit dosage form, but is not limited thereto.

The pharmaceutical composition of the present invention may include tegoprazan, an optical isomer thereof, a pharmaceutically acceptable salt thereof, a hydrate or solvate thereof, or a mixture thereof as the second active ingredient.

Tegoprazan may be a compound represented by formula 1 below, which is also named “(S)-4-(5,7-difluorochroman-4-yloxy)-N,N,2-trimethyl-1H-benzo[d]imidazole-6-carboxamide):”

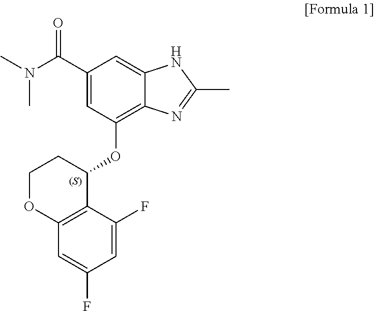

In the present specification, the term “tegoprazan” may refer to tegoprazan, an optical isomer thereof, a pharmaceutically acceptable salt thereof, a hydrate or solvate thereof, or a mixture thereof.

In embodiments of the present invention, the tegoprazan may be in an amorphous or crystalline form. In addition, the pharmaceutically acceptable salt of tegoprazan may be in an amorphous or crystalline form. The pharmaceutical composition of the present invention may stably maintain tegoprazan regardless of the crystallographic form of tegoprazan.

In the present invention, the term “pharmaceutically acceptable salt” may mean the salts formed with any inorganic acid, organic acid or base, which neither causes a serious stimulus to a subject dosed with the pharmaceutical composition containing an active ingredient, nor does damage to biological activity and physical property of the active ingredient. The salts used herein may include the salts conventionally used in the art, such as acid-addition salts formed with pharmaceutically acceptable free acid. The pharmaceutically acceptable salts may be specifically selected from the group consisting of acetate salt, adipate salt, aspartate salt, benzoate salt, besylate salt, bicarbonate salt/carbonate salt, bisulfate salt/sulfate salt, borate salt, camsylate salt, citrate salt, cyclamate salt, edisylate salt, esylate salt, formate salt, fumarate salt, gluceptate salt, gluconate salt, glucuronate salt, hexafluorophosphate salt, hibenzate salt, hydrochloride salt/chloride salt, hydrobromide salt/bromide salt, hydroiodide salt/iodide salt, isethionate salt, lactate salt, malate salt, maleate salt, malonate salt, mesylate salt, methylsulfate salt, naphthylate salt, 2-napsylate salt, nicotinate salt, nitrate salt, orotate salt, palmitate salt, pamoate salt, phosphate salt/hydrogen phosphate salt/dihydrogen phosphate salt, pyroglutamate salt, saccharate salt, stearate salt, succinate salt, tannate salt, tartrate salt, tosylate salt, trifluoroacetate salt, pidolate salt and xinafoate salt, but are not limited thereto. For example, any of the pharmaceutically acceptable salts of tegoprazan may be used without limitation, comprising the above-described salts, as long as they may conventionally show the pharmacological activity of tegoprazan. Specifically, the pharmaceutically acceptable salt of tegoprazan may be tegoprazan pidolate salt or tegoprazan malate salt.

Tegoprazan, which is the second active ingredient, may be effectively used in treating the diseases mediated by an acid pump antagonistic activity, such as gastrointestinal disease, gastroesophageal disease, gastroesophageal reflux disease (GERD), peptic ulcer, gastric ulcer, duodenal ulcer, NSAID-induced ulcer, gastritis, Helicobacter pylori infection, dyspepsia, functional dyspepsia, Zollinger-Ellison syndrome, nonerosive reflux disease (NERD), visceral referred pain, heartburn (pyrosis), nausea, esophagitis, dysphagia, salivation, airway lesion or asthma, in which eligible diseases are not limited to the diseases listed above.

In embodiments of the present invention, the pharmaceutical composition may include the second active ingredient in an amount of about 5 to about 100 mg, specifically about 20 to about 100 mg, and more specifically about 25 to about 50 mg per unit dosage form.

In embodiments of the present invention, the pharmaceutical composition may be for preventing or treating gastrointestinal diseases along with the prevention or treatment of pain and/or inflammatory diseases, and the gastrointestinal diseases may be caused by the use of non-steroidal anti-inflammatory drugs.

In the present invention, the pain and/or inflammatory diseases may include a disease which requires the use of NSAIDs or may be prevented or treated by such use of NSAIDs. For example, it may include cardiovascular disorders, musculoskeletal disorders, acute or chronic diseases with pain and/or inflammation such as migraines, arthralgia, myalgia, rheumatoid arthritis, gout or the like, but are not limited thereto.

In the pharmaceutical composition of the present invention, a non-steroidal anti-inflammatory drug (NSAID), which is the first active ingredient, may be subjected to controlled release, while tegoprazan, an optical isomer thereof, a pharmaceutically acceptable salt thereof, a hydrate or solvate thereof, or a mixture thereof, which is the second active ingredient, may be subjected to immediate release.

In the present invention, the term “immediate release (IR)” may mean that an active ingredient is released immediately or within a short time after administration.

In the present specification, the term “controlled release (CR) or modified release (MR)” or “release control” may mean that the release of a drug is controlled such as the active ingredient rapidly released or continuously released over a long period of time at a specific location of the gastrointestinal tract or after a certain period of time upon taking the drug, and may include both delayed release and/or extended release. Specifically, “controlled release” in the present invention may be a delayed release in which the drug is released at a specific location or after a specific time elapsed after taking the drug, or an extended release in which the drug is slowly released at a specific location or over a certain period of time (for a long time) after a specific time elapsed after taking the drug, or both. More specifically, “controlled release” in the present invention may be a delayed release in which the drug starts to be released under an environment other than gastric juice after taking the drug, or may mean an extended release in which the drug is continuously released under an intestinal environment other than a gastric juice environment after taking the drug. In one specific embodiment, “controlled release” in the present invention may be a delayed release in which the drug starts to be rapidly released under the intestinal environment other than the stomach.

In embodiments of the present invention, the pharmaceutical composition may be one which releases the first active ingredient in a pH range higher than that of gastric juice. Specifically, the first active ingredient in the pharmaceutical composition may not be dissolved or eluted at an acidic pH such as gastric juice, but the first active ingredient may start to be dissolved or eluted in a pH range higher than gastric juice, for example, in a pH range higher than the pH of gastric juice or upper small intestine, for example, in the intestinal fluid environment. More specifically, the first active ingredient may start to be released from the pharmaceutical composition at a pH of about 5 or in a pH range higher than that of. In one specific embodiment, the pharmaceutical composition may be one which releases the first active ingredient under an intestinal (intestinal fluid) environment other than the stomach. Specifically, the first active ingredient in the pharmaceutical composition may be subjected to delayed release or may not be released in a pH range lower than that of the intestinal (intestinal fluid) environment, for example, in an environment such as gastric juice. More specifically, the first active ingredient in the pharmaceutical composition may be released in an amount of less than about 10%, less than about 9%, less than about 8%, less than about 7%, less than about 6%, less than about 5%, less than about 4%, less than about 3%, less than about 2%, or less than about 1% or may not be released in gastric juice or an environment such as the gastric juice. Specifically, the gastric juice or an environment such as the gastric juice may mean an environment having a pH of about 2 to about 4. For example, the pharmaceutical composition may be one which releases the first active ingredient in an amount of less than about 10%, less than about 9%, less than about 8%, less than about 7%, less than about 6%, less than about 5%, less than about 4%, less than about 3%, less than about 2%, or less than about 1%, or does not release the same, within two hours when a dissolution experiment is performed in gastric juice or an acidic environment (e.g., pH of about 2 to about 4).

In embodiments of the present invention, the pharmaceutical composition may be one which releases the second active ingredient in an acidic pH such as gastric juice. In other words, the second active ingredient may start to be dissolved or eluted at an acidic pH such as gastric juice. In one specific embodiment, the pharmaceutical composition may be one which releases the second active ingredient under a gastric acid environment.

When the pharmaceutical composition is administered, non-steroidal anti-inflammatory drug, which is the first active ingredient, may be subjected to controlled release and tegoprazan, which is the second active ingredient, may be subjected to immediate release, and thus the second active ingredient may exhibit a high blood concentration first.

Thus, the pharmaceutical composition of the present invention can exhibit a preventive or therapeutic effect on gastrointestinal diseases, etc., when the pharmaceutical composition is administered. And it is possible to prevent or treat side effects such as symptoms of gastrointestinal disorders or gastrointestinal diseases such as indigestion (gastric pain, heartburn, bloat or nausea), erosion, gastritis/duodenitis, ulcer, gastrointestinal bleeding and the like, and anemia due to excessive bleeding, etc., caused by administering a non-steroidal anti-inflammatory drug which is the first active ingredient.

In addition, the pharmaceutical composition may sufficiently exert a pharmacological effect of each active ingredient without reducing the pharmacological effect of the first active ingredient and the second active ingredient. Specifically, the pharmaceutical composition may exhibit high bioavailability at each level of a single agent without reducing the pharmacological effects of the first active ingredient and the second active ingredient.

Thus, the pharmaceutical composition may exhibit an excellent therapeutic or preventive effect on acute or chronic pain and inflammatory diseases while preventing or treating side effects such as gastrointestinal diseases or symptoms of gastrointestinal disorders or bleeding caused by the administration of the non-steroidal anti-inflammatory drug, which is the first active ingredient, without reducing the pharmacological effects of the first active ingredient and the second active ingredient.

In the present invention, the term “prevention or treatment of gastrointestinal diseases or gastrointestinal disorders” may include preventing, delaying or inhibiting the occurrence of gastrointestinal diseases or gastrointestinal disorders, which are side effects caused by administration of non-steroidal anti-inflammatory drugs, and may include alleviating the symptoms associated with gastrointestinal diseases or gastrointestinal disorders or preventing, delaying, or inhibiting gastrointestinal diseases or gastrointestinal disorders from aggravating.

In the present invention, “administration” may mean providing an effective ingredient (active ingredient) to a subject by any appropriate method, and the pharmaceutical composition of the present invention may be administered via all the general routes, as long as such composition may reach a target tissue. In embodiments of the present invention, the administration may be an oral administration.

In the present invention, the “subject” to which the pharmaceutical composition is administered may include mammals such as humans, guinea pigs, monkeys, cows, horses, sheep, pigs, chickens, turkeys, quails, cats, dogs, mice, rats or rabbits, but is not limited thereto, and may be specifically humans.

In embodiments of the present invention, the pharmaceutical composition may be a pharmaceutical combination preparation, and specifically may be a combination preparation in which the first active ingredient and the second active ingredient are formulated into one unit dosage form. In this case, the first active ingredient and the second active ingredient may be included together in one unit dosage form. For example, when the unit dosage form is a tablet, the tablet may include both the first active ingredient and the second active ingredient.

In one specific embodiment, the pharmaceutical composition may be pharmaceutical combination preparation which includes the first compartment containing the first active ingredient and the second compartment containing the second active ingredient, in which the first compartment and the second compartment are formulated into one unit dosage form, and the first active ingredient may be subjected to controlled release from the pharmaceutical combination preparation and the second active ingredient may be subjected to immediate release from the pharmaceutical combination preparation.

Specifically, the first active ingredient may be subjected to delayed release from the pharmaceutical combination preparation. In one specific embodiment, the pharmaceutical composition (the pharmaceutical combination preparation) may be one in which the first active ingredient is subjected to delayed release under an intestinal (intestinal fluid) environment.

In embodiments of the present invention, the unit dosage form of the pharmaceutical composition, which is the combination preparation, may be a tablet or a capsule, and the tablet may be a tablet-in-tablet, a coated tablet (multi-coated tablet), or a multi-layered tablet (two-layer tablet or three-layer tablet) having a laminate structure.

In the unit dosage form, the first compartment containing the first active ingredient may include a particle containing: a core having the first active ingredient; and an enteric coated layer located on the core and surrounded the core, and the second compartment containing the second active ingredient may include a particle containing the second active ingredient or a second active ingredient layer containing the second active ingredient.

In the present specification, the compartment may mean a region containing an active ingredient, such as a particle containing the active ingredient, a layer containing the particle, or a layer containing the active ingredient in the pharmaceutical composition of the present invention, and may represent the particle per se, a layer containing the particle, or a layer containing the active ingredient depending on the unit dosage form of the pharmaceutical composition.

In embodiments of the present invention, when the unit dosage form of the pharmaceutical composition, which is the combination preparation, is a tablet, the tablet may include the first compartment containing the first active ingredient and the second compartment containing the second active ingredient and located on the first compartment, which surrounded the first compartment or laminated in the first compartment.

Specifically, the first compartment may include a particle containing: a core having the first active ingredient; and an enteric coated layer located on the core and surrounded the core. The first compartment may mean the particle per se or may mean a layer containing the particle depending on a tablet shape. In the present invention, the particle may be referred to as a particle containing the first active ingredient.

The enteric coated layer may be an enteric coated layer containing an enteric material that is soluble in a pH dependent way. Specifically, the enteric coated layer containing the enteric material which is soluble in a pH dependent way may not dissolve in an acidic pH such as the stomach, but may dissolve at a pH of about 5 or in a pH range higher than that of, such as an intestinal environment. For example, the enteric coated layer may be insoluble at an acidic pH of the environment such as the stomach, but may be soluble in a pH range of the environment such as the intestine. More specifically, the enteric coated layer may not dissolve at all or hardly dissolve (about 10% or less) at a pH of less than about 5, and then rapidly dissolve at a pH of about 5 or higher (about pH 5.5, 6, 6.5, 7). Thus, in the pharmaceutical composition of the present invention, the first active ingredient may not be released at all from the pharmaceutical composition (e, g. the particle containing the first active ingredient) at a pH of less than about 5 (e.g., gastric juice environment) or may be hardly released, but may be released at a pH of about 5 or higher (e.g., intestinal fluid environment). In other words, when the pharmaceutical composition is administered to a subject, the first active ingredient may not be released from the pharmaceutical composition under a gastric juice environment (acid resistance), but may be rapidly released or slowly released over a certain period of time in an intestinal fluid environment. Specifically, the first active ingredient may be rapidly released under the intestinal fluid (intestinal) environment.

In embodiments of the present invention, the enteric material included in the enteric coated layer may be soluble at a pH of about 5 or higher, specifically soluble at a pH of about 5 to about 7.5, and may include at least one selected from, for example, methacrylic acid copolymer, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac, and other appropriate functional materials. Specifically, the enteric layer material may include a methacrylic acid copolymer.

In embodiments of the present invention, the enteric coated layer may be prepared by using a solution in which the enteric material is dispersed or dissolved in an appropriate solvent. In this case, the solvent may be water, an organic solvent, or a mixture thereof, specifically may be water, C₁ to C₅ straight or branched chain alcohol, acetone, or a mixture thereof, more specifically may be water, methanol, ethanol, isopropyl alcohol, acetone, or a mixture thereof, and much more specifically may be water, ethanol, or an aqueous ethanol solution.

In embodiments of the present invention, the solution in which the enteric material is dispersed or dissolved in an appropriate solvent may further include a pharmaceutically acceptable plasticizer in order to achieve desired mechanical properties such as flexibility and hardness of the enteric coated layer. Accordingly, the enteric coated layer may further include the plasticizer. The plasticizer may include, for example, triacetin, citric acid ester, phthalic acid ester, dibutyl sebacate, cetyl alcohol, polyethylene glycol, polysorbate or other plasticizers, but is not limited thereto. The amount of the plasticizer may be optimized in such a way that mechanical properties of the enteric coated layer(s), i.e. flexibility and hardness, which are exemplified by Vickers hardness, for example, are adjusted not to remarkably reduce the acid resistance of the core covered with the enteric coated layer with respect to the enteric material(s) and plasticizer(s) selected and the amount of the enteric materials(s) applied to form the enteric coated layer when formulating the core (for example, when compressing into a tablet). In addition, in embodiments of the present invention, additives such as dispersants, colorants, pigment polymers, for example poly(ethyl acrylate, methyl methacrylate), anti-viscosity agents and anti-foaming agents may be included in the enteric coated layer. Furthermore, in embodiments of the present invention, other compounds may be added to increase a layer thickness and reduce the diffusion of acidic gastric juice into an acid-sensitive material.

In embodiments of the present invention, a solution in which the enteric material is dispersed or dissolved in an appropriate solvent may include triethyl citrate, polysorbate 80, or a mixture thereof in order to form the enteric coated layer.

In embodiments of the present invention, the enteric coated layer may be about 5 wt % or more, preferably about 6 wt % or more, about 7 wt % or more, about 8 wt % or more, about 9 wt % or more, more preferably about 10 wt % or more based on the total weight of the core containing the first active ingredient, but is not necessarily limited thereto, in order to protect NSAID, which the first active ingredient, and obtain satisfactory acid resistance of the form (formulation) of the pharmaceutical composition according to the present invention. In embodiments of the present invention, the thickness of the enteric coated layer may be appropriately adjusted depending on conventional processing conditions and a desired dissolution profile. For example, the enteric coated layer may be about 20 wt % or less and about 15 wt % or less based on the total weight of the core containing the first active ingredient, but is not necessarily limited thereto.

In embodiments of the present invention, the particle containing the first active ingredient may be in the form of tablet, granule, or pellet.

In one specific embodiment, when the particle containing the first active ingredient is tablet, the core may be an uncoated tablet containing granule having the first active ingredient. The uncoated tablet may further contain pharmaceutically acceptable additive, and the pharmaceutically acceptable additive may be contained inside the granule, outside or inside the granule, or outside the granule. In this case, an enteric coated layer located on the uncoated tablet and surrounded the uncoated tablet may be formed. The granule may be wet granule, direct compression granule, or mixture thereof, and the granule may be one which has been subjected to wet granulation by using a high-speed shear granulator or which has been subjected to direct compression granulation by using a fluidized bed granulator. Specifically, the granule may be a wet granule prepared by wet granulation in order to improve tableting properties of the NSAID. The wet granulation may be performed by using a solution selected from the group consisting of water, ethanol, isopropanol, and a mixture thereof, and specifically water may be preferable, but is not limited thereto.

In another specific embodiment, when the particle containing the first active ingredient is granule, the core may be initial granule containing the first active ingredient. The granule may further include pharmaceutically acceptable additives. In this case, the granule may be in a form surrounded by an enteric coating material. The granule may be substantially the same as described for the granule included in the uncoated tablet, if not contradictory to each other.

In another specific embodiment, when the particle containing the first active ingredient is a pellet, the core may include a seed and a coated layer located on the seed and containing the first active ingredient. The seed may be a sugar seed that does not contain a pharmaceutically active ingredient, and the coated layer containing the first active ingredient may further include pharmaceutically acceptable additive. In this case, an enteric coated layer located on the pellet and surrounded the pellet may be formed.

In the present invention, the term “pharmaceutically acceptable additive” may be a material that neither irritates organisms nor inhibits the biological activity and properties of injected (administered) active ingredient, and the type of the additive that can be used in the present invention is not particularly limited and any additive may be used as long as it is conventionally used in the art and pharmaceutically acceptable. For example, the pharmaceutically acceptable additive may include a pharmaceutically acceptable excipient, etc., selected from the group consisting of carriers, binders, disintegrants, lubricants, and mixtures thereof, and may further include any other additives and adjuvants such as plasticizers, colorants, pigments, fillers, anti-viscosity agents and anti-static agents.

The binder may be selected from the group consisting of alginic acid, sodium alginate, sodium carboxymethylcellulose, ethylcellulose, hydroxyethylcellulose, hydroxypropylcellulose, hydroxypropylmethylcellulose, methylcellulose, gelatin, povidone, starch, pregelatinized starch and mixtures thereof, but is not limited thereto. The binder may be included in an amount of about 1 to about 20 wt %, preferably about 1 to about 5 wt % based on the total weight of the particles.

The disintegrant may be selected from the group consisting of alginic acid, sodium alginate, sodium carboxymethylcellulose, microcrystalline cellulose, powdered cellulose, croscarmellose sodium, crospovidone, pregelatinized starch, sodium starch glycolate, starch and mixtures thereof, but is not limited thereto. The disintegrant may be included in an amount of about 0.5 to about 10 wt %, preferably about 1 to about 5 wt % based on the total weight of the particles.

The lubricant may be selected from the group consisting of calcium stearate, glyceryl monostearate, glyceryl palmitostearate, magnesium stearate, sodium lauryl sulfate, sodium stearyl fumarate, zinc stearate, stearic acid, hydrogenated vegetable oil, polyethylene glycol, benzoic acid sodium, tale and mixtures thereof, but is not limited thereto. The lubricant may be included in an amount of about 0.2 to about 5 wt %, preferably about 1 to about 3 wt % based on the total weight of the particles.

Any of the other additives and adjuvants may be diluents, colorants, anti-adhesive agents, or mixtures thereof, but are not limited thereto. For example, any other additives and adjuvants may be, for example, magnesium stearate, titanium dioxide, tale, and other additives.

The diluent may be selected from the group consisting of mannitol, sucrose, microcrystalline cellulose, lactose, sorbitol, xylitol, glucose, and mixtures thereof, but is not limited thereto.

The core may contain the first active ingredient in an amount of about 20 to about 95 wt %, preferably about 50 to about 95 wt % based on the total weight of the core.

In the present invention, “coating” may mean coating (applying) or laminating by a conventional coating or lamination procedure, but is not limited thereto. For example, the coating may mean applying or laminating by a coating or lamination procedure in a suitable apparatus such as a coating pan and a coating granulator, or in a fluidized bed apparatus in which water and/or organic solvents are used in a coating or lamination process. In the present invention, “coated layer” may mean a layer formed by coating.

In embodiments of the present invention, the particle containing the first active ingredient may further include a separation layer. The separation layer may be located between the core containing the first active ingredient and the enteric coated layer of the particle, or located on the enteric coated layer, or located both between the core and the enteric coated layer and on the enteric coated layer. The separation layer may perform a function of an isolation layer that reliably blocks a contact with substances that may affect the stability of the first active ingredient, and may prevent the core containing the first active ingredient and the particle containing the same from being cracked or deformed. Thus, it is possible to enhance the stability of the first active ingredient and the particle containing the same. In the present invention, the separation layer may be interchangeably referred to as an isolation layer or barrier.

In embodiments of the present invention, the separation layer may be a layer that is water-soluble or rapidly disintegrates in water. The separation layer may include all the means applicable to oral dosage forms by those skilled in the art, such as membranes, walls, coatings, etc., made of the separation layer material which is water-soluble or rapidly disintegrates in water. For example, the separation layer may be formed by applying the separation layer material according to a coating or lamination procedure in a suitable apparatus such as a coating pan and a coating granulator, or in a fluidized bed apparatus in which water and/or organic solvents are used in a coating or lamination process. The separation layer material forming the separation layer may be at least one selected from pharmaceutically acceptable compounds, such as sugar, polyethylene glycol, polyvinylpyrrolidone, polyvinyl alcohol, polyvinyl acetate, hydroxypropyl cellulose, methylcellulose, ethylcellulose, hydroxypropylmethylcellulose, sodium carboxymethylcellulose, etc. The separation layer may contain additives such as plasticizers, colorants, pigments, fillers, anti-viscosity agents and antistatic agents, for example, magnesium stearate, titanium dioxide, tale, and other additives. The separation layer may form a separation layer by dispersing or dissolving a pharmaceutically acceptable additive in a solvent and coating on the core and/or the enteric coated layer. Pharmaceutically acceptable additives and solvents may be substantially the same as described above, if not contradictory to each other. The separation layer may prevent the core and the particle from being cracked and may separate the core from the enteric coated layer, and thus may further enhance the stability of the pharmaceutical composition of the present invention.

In addition, the second compartment may include a second active ingredient layer located on the first compartment and containing the second active ingredient.

In embodiments of the present invention, when the unit dosage form is a tablet in tablet or a coated tablet in the pharmaceutical composition, which is the combination preparation, the second active ingredient layer may be located on and surround the first compartment. In this case, the first compartment may be the particle per se containing the first active ingredient, and specifically the particle containing the first active ingredient may be a tablet.

The second active ingredient layer may be formed by coating a composition containing the second active ingredient on the particle (first compartment) containing the first active ingredient to surround the particle containing the first active ingredient, or may be formed by tableting a mixture containing: granules containing the second active ingredient and pharmaceutically acceptable additives; and pharmaceutically acceptable additives other than the granules, together with the particles (first compartment) containing the first active ingredient. In this case, the granule may be a wet granule or a direct compression granule.

In embodiments of the present invention, the second active ingredient layer may be a coated layer surrounding the particles (first compartment) containing the first active ingredient, and may be formed with a coating solution containing the second active ingredient and pharmaceutically acceptable additives. Specifically, the second active ingredient layer may be formed by applying the coating solution on the particles (first compartment) containing the first active ingredient to surround the particles (first compartment) containing the first active ingredient by an appropriate coating or lamination method.

Any of the additives may be used without a particular limitation as long as they are conventionally used in the art and pharmaceutically acceptable. A non-limiting example of the additives may be at least one selected from pharmaceutically acceptable compounds, such as sugar, polyethylene glycol, polyvinylpyrrolidone, polyvinyl alcohol, polyvinyl acetate, hydroxypropyl cellulose, methylcellulose, ethylcellulose, hydroxypropylmethylcellulose, sodium carboxymethylcellulose, etc. In addition, the additives may be substantially the same as described for the pharmaceutically acceptable additives, if not contradictory to each other.

In one specific embodiment, when the particle containing the first active ingredient includes separation layer(s), and when the separation layer is located on the enteric coated layer in the particle containing the first active ingredient, the second active ingredient layer may be formed on the separation layer. In another specific embodiment, when the particle containing the first active ingredient includes separation layer, but with the separation layer being present only between the core and the enteric coated layer, that is, when the separation layer in the particle containing the first active ingredient is not located on the enteric coated layer, the second active ingredient layer may be located on the enteric coated layer of the particle containing the first active ingredient.

In embodiments of the present invention, the separation layer may be present between the particle (first compartment) containing the first active ingredient and the second active ingredient layer (second compartment), and in this case, the separation layer may block a contact between the first active ingredient and the second active ingredient, and separate the enteric coated layer of the first compartment and the second compartment.

The pharmaceutical composition of the present invention can maintain excellent stability of active ingredients because the first active ingredient and the second active ingredient do not come into contact with each other by the enteric coated layer or the separation layer formed on the enteric coated layer. In particular, when including the separation layer, it is possible to prevent particles from being split or cracked so as to provide excellent formulation stability.

In embodiments of the present invention, a colored layer may be further included on the second active ingredient layer.

The colored layer may be located on the second active ingredient layer containing the second active ingredient, and may be a layer surrounding the second active ingredient layer and a layer which is water-soluble or rapidly disintegrates or dissolves in water. The colored layer may be applied by a coating or lamination procedure in a suitable apparatus such as a coating pan and a coating granulator, or in a fluidized bed apparatus in which water and/or organic solvents are used in a coating or lamination process. The colored layer may be formed from a colored coating substrate such as opadry and/or at least one selected from pharmaceutically acceptable compounds, such as sugar, polyethylene glycol, polyvinylpyrrolidone, polyvinyl alcohol, polyvinyl acetate, hydroxypropyl cellulose, methylcellulose, ethylcellulose, hydroxypropylmethylcellulose, sodium carboxymethylcellulose, etc., which are used alone or in mixture. In addition, the colored layer may contain additives such as plasticizers, colorants, pigments, fillers, anti-viscosity agents and antistatic agents, for example, magnesium stearate, titanium dioxide, talc, and other additives.

In embodiments of the present invention, when the unit dosage form is a multi-layered tablet in the pharmaceutical composition, which is combination preparation, the multi-layered tablet may be one, in which the second active ingredient layer containing particles having the second active ingredient (second compartment) is laminated on the first compartment. In this case, the particle may be the granule. In this case, the first compartment may be a layer containing particles having the first active ingredient. Specifically, the multi-layered tablet may be formed by tableting the particles containing the first active ingredient, which are the granules, and the granules including the second active ingredient and pharmaceutically acceptable additives by using a multi-layer tablet press, and the granule may be wet granule or direct compression granule.

The multi-layered tablet may further include a separation layer between the first compartment and the second compartment, and the separation layer may be substantially the same as described above, if not contradictory to each other.

The multi-layered tablet may further include a colored layer, and the colored layer may surround the multi-layered tablet in which the first compartment and the second compartment are laminated. The colored layer may be substantially the same as described above, if not contradictory to each other.

In embodiments of the present invention, when the unit dosage form of the pharmaceutical composition, which is combination preparation, is a capsule, the capsule may be a capsule filled with the tablet of the present invention described above. In this case, the tablet may be mini-tablet. Specifically, the capsule may be a capsule filled with the mini-tablet of the tablet of the present invention described above.

In another specific embodiment, the capsule may be a capsule filled with particles containing the first active ingredient; and particles containing the second active ingredient. Specifically, the particles containing the first active ingredient and the particles containing the second active ingredient may be each independently tablet, granule, pellet, or mixture thereof.

The particles containing the first active ingredient may be substantially the same as the particles containing the first active ingredient described in the case where the unit dosage form is a tablet, if not contradictory to each other.

When the particle containing the second active ingredient is a tablet, the core may be an uncoated tablet containing granule having the second active ingredient. The uncoated tablet may further contain pharmaceutically acceptable additive, and the pharmaceutically acceptable additive may be contained inside the granule and/or outside the granule.

When the particle containing the second active ingredient is granule, the core may be initial granule containing the second active ingredient. The granule may further include pharmaceutically acceptable additives.

When the particle containing the second active ingredient is a pellet, the core may include a seed and a coated layer located on the seed and containing the second active ingredient. The pellet may further include pharmaceutically acceptable additives.

The pharmaceutically acceptable additives in the particle containing the second active ingredient may be substantially the same as described for the particle containing the first active ingredient, if not contradictory to each other.

The particle containing the second active ingredient may further include a separation layer on the particle, and the separation layer may be substantially the same as described for the particle containing the first active ingredient, if not contradictory to each other.

The particle containing the first active ingredient and/or the particle containing the second active ingredient may further include a colored layer located on the particle containing the first active ingredient and/or the particle containing the second active ingredient and surrounded the particle, and the colored layer may be substantially the same as described above, if not contradictory to each other.

In embodiments of the present invention, it may be preferable that the unit dosage form in the pharmaceutical composition as the combination preparation is a coated tablet (multi-coated tablet).

In one specific embodiment, the unit dosage form may include: the core containing the first active ingredient; the enteric coated layer located on the core and surrounded the core; and the second active ingredient layer, which is coated layer located on the enteric coated layer and surrounded the enteric coated layer and containing the second active ingredient. In this case, the core may be in an uncoated tablet form.

In another specific embodiment, the unit dosage form may include: the core containing the first active ingredient; the separation layer located on the core and surrounded the core; the enteric coated layer located on the separation layer and surrounded the separation layer; and the second active ingredient layer, which is coated layer located on the enteric coated layer and surrounded the enteric coated layer and containing the second active ingredient. In this case, the core may be an uncoated tablet.

In another specific embodiment, the unit dosage form may include: the core containing the first active ingredient; the enteric coated layer located on the core and surrounded the core; the separation layer located on the enteric coated layer and surrounded the enteric coated layer; and the second active ingredient layer, which is coated layer located on the separation layer and surrounded the separation layer and containing the second active ingredient. In this case, the core may be an uncoated tablet.

In another specific embodiment, the unit dosage form may include: the core containing the first active ingredient; a first separation layer located on the core and surrounded the core; the enteric coated layer located on the first separation layer and surrounded the first separation layer; a second separation layer located on the enteric coated layer and surrounded the enteric coated layer; and the second active ingredient layer, which is coated layer located on the second separation layer and surrounded the second separation layer and containing the second active ingredient. In this case, the core may be an uncoated tablet.

In the specific embodiments of the present invention, the unit dosage form may further include a colored layer located on the second active ingredient layer and surrounded the second active ingredient layer.

In the pharmaceutical composition as the combination preparation of the present invention, from the unit dosage form, the first active ingredient may be subjected to controlled release, and the second active ingredient may be subjected to immediate release. Specifically, from the unit dosage form, the first active ingredient may be rapidly released in the intestinal (intestinal fluid) environment (delayed release), and the second active ingredient may be released in the gastric (gastric fluid) environment. Thus, when administered, the pharmaceutical composition may exhibit an excellent therapeutic effect on pain and inflammation with the non-steroidal anti-inflammatory drug as the first active ingredient, and also prevent or treat side effects such as symptoms of gastrointestinal disorders or gastrointestinal diseases such as indigestion (gastric pain, heartburn, bloat or nausea), erosion, gastritis/duodenitis, ulcer, gastrointestinal bleeding and the like, and anemia, etc., due to excessive bleeding caused by the administration of the non-steroidal anti-inflammatory drug, thereby minimizing the side effects. In particular, even when a subject with chronic pain and inflammatory disease requiring continuous treatment with a non-steroidal anti-inflammatory drug continues to take the non-steroidal anti-inflammatory drug, it is also possible to prevent the recurrence of side effects caused by taking non-steroidal anti-inflammatory drugs while treating the side effects caused by taking non-steroidal anti-inflammatory drugs.

In addition, the pharmaceutical composition of the present invention contains non-steroidal anti-inflammatory drug as the first active ingredient and tegoprazan as the second active ingredient in one unit dosage form, and thus shows more excellent convenience of and compliance to medication than separate administration of the active ingredients.

Furthermore, in spite of including both the first active ingredient and the second active ingredient in one unit dosage form, the pharmaceutical composition may show excellent stability and excellent pharmacological effects without deterioration of the medicinal efficacy of the active ingredients.

The present invention may provide a method for preparing the pharmaceutical composition of the present invention.

The method for preparing the pharmaceutical composition of the present invention may include:
forming a first compartment containing the first active ingredient; and
forming a second compartment containing the second active ingredient on the first compartment.

In embodiments of the present invention, in the method for preparing the pharmaceutical composition, the forming of the first compartment containing the first active ingredient may include preparing a particle containing the first active ingredient, and the preparing of a particle containing the first active ingredient may include:
forming a core containing the first active ingredient; and
forming an enteric coated layer on the core.

In the forming of the enteric coated layer, there may be formed the particle containing the core containing the first active ingredient and the enteric coated layer located on the core and surrounded the core.

The particle may be tablet, granule or pellet, and the particle, the tablet, the granule, the pellet and preparation thereof may be substantially the same as described for the pharmaceutical composition, if not contradictory to each other. For example, when the particle is a tablet, an uncoated tablet may be prepared by tableting granules containing the first active ingredient, and the granules may be prepared from a mixture of the first active ingredient and pharmaceutical acceptable additives by wet granulation using a high-speed shear granulator or by direct compression granulation using a fluidized bed granulator. Specifically, the granules may be prepared by wet granulation.

In embodiments of the present invention, in the preparing of the particle containing the first active ingredient, forming a separation layer may be further included before and/or after the forming of an enteric coated layer on the core.

When the forming of the separation layer is further included before the forming of the enteric coated layer, the enteric coated layer may be formed on the core on which the separation layer is formed. When the forming of the separation layer is further included after the forming of the enteric coated layer, the separation layer may be formed on the enteric coated layer.

In embodiments of the present invention, the forming of the second compartment containing the second active ingredient may include coating the composition containing the second active ingredient on the first compartment. Specifically, there may be included coating the composition containing the second active ingredient on the particle containing the core having the first active ingredient and containing the enteric coated layer located on the core and surrounded the core. When the particle further includes the separation layer between the core and the enteric coated layer and/or on the enteric coated layer, there may be included coating the composition containing the second active ingredient on the enteric coated layer on which the separation layer is formed.

In embodiments of the present invention, there may be further included coating a colored layer after the coating of the composition containing the second active ingredient on the first compartment. The colored layer may be substantially the same as described above.

In one specific embodiment, the method for preparing the pharmaceutical composition of the present invention may include:
forming a core containing the first active ingredient;
forming an enteric coated layer on the core and surrounded the core; and
forming a second active ingredient layer (a second active ingredient coated layer) containing the second active ingredient on the enteric coated layer and surrounded the enteric coated layer.

In another specific embodiment, the method for preparing the pharmaceutical composition of the present invention may include:
forming a core containing the first active ingredient;
forming a separation layer on the core; and
forming an enteric coated layer on the separation layer and surrounded the separation layer; and
forming a second active ingredient layer (a second active ingredient coated layer) containing the second active ingredient on the enteric coated layer and surrounded the enteric coated layer.

In still another specific embodiment, the method for preparing the pharmaceutical composition of the present invention may include:
forming a core containing the first active ingredient;
forming an enteric coated layer on the core and surrounded the core;
forming a separation layer on the enteric coated layer and surrounded the enteric coated layer; and
forming a second active ingredient layer (a second active ingredient coated layer) containing the second active ingredient on the separation layer and surrounded the separation layer.

In still another specific embodiment, the method for preparing the pharmaceutical composition of the present invention may include:
forming a core containing the first active ingredient;
forming a first separation layer on the core;
forming an enteric coated layer on the first separation layer and surrounded the first separation layer;
forming a second separation layer on the enteric coated layer and surrounded the enteric coated layer; and
forming a second active ingredient layer (a second active ingredient coated layer) containing the second active ingredient on the second separation layer and surrounded the second separation layer.

In the above specific embodiments, there may be further included forming a colored layer on the second active ingredient layer and surrounded the second active ingredient layer.

In the above specific embodiments, the core may be uncoated tablet, and the forming of the core containing the first active ingredient may be tableting granules containing the first active ingredient to form a core, which is uncoated tablet containing the first active ingredient.

In the method for preparing the pharmaceutical composition of the present invention, the pharmaceutical composition, the first active ingredient, the second active ingredient, the core, the enteric coated layer, the separation layer, the second active ingredient layer, and the colored layer may be the same as described for the pharmaceutical composition, if not contradictory to each other.

The present invention may provide a method for preventing or treating inflammatory diseases, pain diseases, and/or gastrointestinal diseases, including administering the pharmaceutical composition of the present invention.

In the method for preventing or treating of the present invention, a pharmaceutically effective amount of the pharmaceutical composition may be administered to a subject in need thereof.

In the present invention, “pharmaceutically effective amount” may mean an amount enough to treat a disease at a reasonable benefit/risk ratio applicable to medical treatment, and a level of effective amount may be determined according to factors including a patient's disease type, severity, activity of a drug, sensitivity to the drug, an administration time, an administration route and excretion rate, a treatment period and a concurrently used drug, as well as other factors well known in a medical field. Considering all the factors described above, it is important to carry out an administration by an amount, in which the maximum effect may be achieved by the minimum amount without a side effect, in which such amount may be easily determined by those skilled in the art.

In the method for preventing or treating of the present invention, the pharmaceutical composition may be substantially the same as described for the pharmaceutical composition of the present invention. In addition, the subject and administration may be the same as described for the pharmaceutical composition of the present invention.

The present invention may provide a use of the pharmaceutical composition of the present invention for preventing or treating inflammatory diseases, pain diseases and/or gastrointestinal diseases.

The present invention may provide a use of the pharmaceutical composition of the present invention in preparation of a drug for preventing or treating inflammatory diseases, pain diseases and/or gastrointestinal diseases.

In the above uses, the pharmaceutical composition may be substantially the same as described for the pharmaceutical composition of the present invention.

### Advantageous Effects

The pharmaceutical composition of the present invention may include non-steroidal anti-inflammatory drug and tegoprazan in one unit dosage form, so as to enhance the convenience of and compliance to medication, improve the stability of active ingredients and preparations, and maintain tegoprazan in the form of immediate release while maintaining the controlled release of the non-steroidal anti-inflammatory drug, thereby exhibiting bioavailability equivalent to the case of co-administration of each thereof. Thus, it is possible to remarkably ameliorate and minimize the side effects of the gastrointestinal system caused by the existing non-steroidal anti-inflammatory drug.

### BRIEF DESCRIPTION OF THE DRAWINGS

FIG. 1 is a mimetic view showing a tablet according to one embodiment of the present invention.

FIGS. 2 to 4 are views showing DSC results of tegoprazan crystalline, amorphous and polymorphic forms in a tegoprazan layer containing tegoprazan crystalline, amorphous and polymorphic crystal forms. In FIGS. 2 to 4, “initial” represent the results of storage at an initial stage and “stress weeks 4” represent the results after storage for four weeks under stress conditions, respectively.

FIG. 5 is a view showing the results of in-vivo pharmacokinetic parameters of the tablet according to one specific embodiment of the present invention. In A and B of FIG. 5, references show the results of 25 mg of K-CAB tablet and 500 mg of Naprosyn EC, each of which is a co-administration control drug.

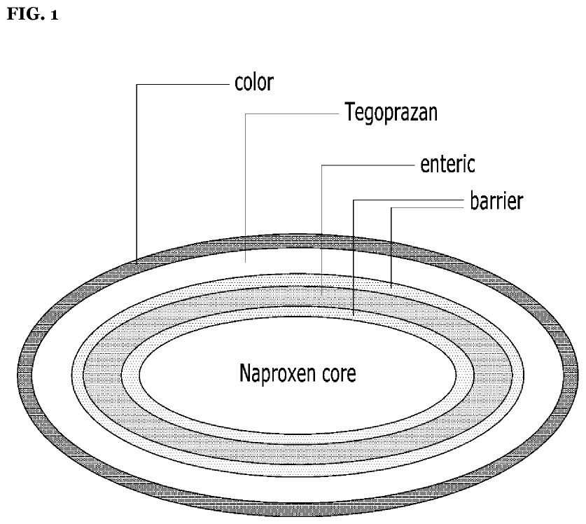

### MODE FOR INVENTION

Hereinafter, the present invention will be described with reference to examples (embodiments). However, the following exemplary examples (embodiments) are provided only for the purpose of illustrating the present invention, and thus the present invention is not limited thereto.

### Examples 1 to 5: Preparation of Core Tablet

A core tablet was prepared by using the composition described in table 1 below. Specifically, with a binder solution obtained by dissolving povidone, hydroxypropylcellulose and hydroxypropylmethylcellulose in purified water, a mixture of naproxen and croscarmellose sodium or a mixture of naproxen and crospovidone was subjected to wet granulation using a high-speed shear granulator or granulation using a fluidized bed granulator. Then, colloidal silicon dioxide and magnesium stearate were added and mixed to prepare a mixture for preparing a naproxen core (uncoated tablet). The mixture was compressed into tablets by using a tablet press (Sejong Pharmatech).

TABLE 1

| Classification | | Example 1 | Example 2 | Example 3 | Example 4 | Example 5 |
|---|---|---|---|---|---|---|
| Uncoated | Granulation | High speed shear granulation | | | | Fluidized bed |
| core layer | Method | | | | | granulation |
| | Naproxen | 500.0 | 500.0 | 500.0 | 500.0 | 500.0 |
| | Povidone | 10.0 | — | — | 10.0 | 10.0 |
| | Hydroxypropyl- | — | 10.0 | — | — | — |
| | cellulose | | | | | |
| | Hydroxypropyl- | — | — | 10.0 | — | — |
| | methylcellulose | | | | | |
| | Colloidal | 3.0 | 3.0 | 3.0 | 3.0 | 3.0 |
| | silicon | | | | | |
| | dioxide | | | | | |
| | Croscarmellose | 30.0 | 30.0 | 30.0 | — | 30.0 |
| | sodium | | | | | |
| | Crospovidone | — | — | — | 30.0 | — |
| | Magnesium | 1.5 | 1.5 | 1.5 | 1.5 | 1.5 |
| | stearate | | | | | |
| | Purified water | 200.0 | 200.0 | 200.0 | 200.0 | 500.0 |
| | Total weight | 544.5 | 544.5 | 544.5 | 544.5 | 544.5 |

(Unit: mg/tablet)

The total weight in the table above excludes the solvent weight.

### Test Example 1: Naproxen Dissolution Test

A naproxen dissolution test was performed in 900 mL of a pH 7.4 solution with respect to the core tablets of Examples 1 to 5 and Naxen F tablet 500 mg (control drug, Chong Kun Dang) according to the paddle method. The test solutions were collected at 5, 10, 15, 30, 45 and 60 minutes after starting the test, a dissolution rate of naproxen was confirmed by using high performance liquid chromatography, and the specific conditions are as follows.

### <High Performance Liquid Chromatography Conditions>

Column: A column filled with 5 μm of octadecylsilyl silica gel for liquid chromatography in a stainless steel tube with an inner diameter of about 4.6 mm and a length of about 15 cm or a column equivalent thereto
Column temperature: Constant temperature around 30° C.
Detector: Ultraviolet absorption spectrophotometer (wavelength: 262 nm)
Flux: 1.0 mL/min
Mobile phase: Mixture of 0.01 mol/L ammonium acetate buffer: acetonitrile (11:9)
(Prepared by dissolving 0.01 mol/L ammonium acetate buffer: ammonium acetate (CH3COONH4) 0.77 g in 1 L of water and filtering through a 0.45 μm membrane filter)
Eluate: pH 7.4 solution, 900 mL, paddle method, 75 rpm

The results of confirming the dissolution rates of naproxen from the core tablets of Examples 1 to 5 and Naxen F 500 mg are shown in table 2.

TABLE 2
Naproxen dissolution rate (%)

| Time (min) | Example 1 | Example 2 | Example 3 | Example 4 | Example 5 | Naxen F tablet |
|---|---|---|---|---|---|---|
| 0 | 0.0 | 0.0 | 0.0 | 0.0 | 0.0 | 0.0 |
| 5 | 80.9 | 77.5 | 88.9 | 78.5 | 85.6 | 82.1 |
| 10 | 97.6 | 87.6 | 99.5 | 96.5 | 99.0 | 98.5 |
| 15 | 100.0 | 95.1 | 99.7 | 99.8 | 100.1 | 99.9 |
| 30 | 99.9 | 96.7 | 100.0 | 99.9 | 100.2 | 100.1 |
| 45 | 100.1 | 98.9 | 100.1 | 99.9 | 100.2 | 100.2 |
| 60 | 100.1 | 99.0 | 100.1 | 100.0 | 100.4 | 100.2 |

As shown in above table 2, there was no difference in dissolution rate according to granule preparation methods as shown in the results of the core tablet of Example 1 prepared by using the granules prepared by high-speed shear granulation, and the core tablet of Example 5 prepared by using the granules prepared by using the fluidized bed granulation, and it can be confirmed that there is no difference in dissolution rate according to the composition of tablets as shown in Examples 1 to 4.

### Examples 6 to 12: Application of Separation Layer and Enteric Layer

A separation layer and an enteric layer with the composition of table 3 below were applied to the core tablet prepared according to Example 1. Specifically, a primary separation layer was coated and laminated on the surface of the core tablet prepared according to Example 1 by using a hydroxypropyl methylcellulose (HPMC) or ethyl cellulose (EC) solution prepared as shown in the composition of table 3 below. An enteric layer was coated and laminated thereon by using a solution prepared from methacrylic acid copolymer L30 D-55 (trade name Eudragit® L30 D-55; manufactured by EVONIK Industries) (Examples 6 to 11). Example 12 was prepared by forming the enteric layer directly on the surface of the core tablet. For reference, in embodiments of the present invention, the amount of methacrylic acid copolymer L30 D-55 (trade name Eudragit® L30 D-55; manufactured by EVONIK Industries) means the amount as a solid content.

TABLE 3

| Classification | | Example 6 | Example 7 | Example 8 | Example 9 | Example 10 | Example 11 | Example 12 |
|---|---|---|---|---|---|---|---|---|
| Uncoated | Naproxen | 500.0 | 500.0 | 500.0 | 500.0 | 500.0 | 500.0 | 500.0 |
| core layer | Povidone | 10.0 | 10.0 | 10.0 | 10.0 | 10.0 | 10.0 | 10.0 |
| | Colloidal | 3.0 | 3.0 | 3.0 | 3.0 | 3.0 | 3.0 | 3.0 |
| | silicon | | | | | | | |
| | dioxide | | | | | | | |
| | Croscarmellose | 30.0 | 30.0 | 30.0 | 30.0 | 30.0 | 30.0 | 30.0 |
| | sodium | | | | | | | |
| | Magnesium | 1.5 | 1.5 | 1.5 | 1.5 | 1.5 | 1.5 | 1.5 |
| | stearate | | | | | | | |
| | Purified | 200.0 | 200.0 | 200.0 | 200.0 | 200.0 | 200.0 | 200.0 |
| | water | | | | | | | |
| Primary | Hydroxypropyl | 5.0 | — | — | 5.0 | 5.0 | — | — |
| separation | methylcellulose | | | | | | | |
| layer | 3 cps | | | | | | | |
| | Hydroxypropyl | — | 5.0 | — | — | — | — | — |
| | methylcellulose | | | | | | | |
| | 4.5 cps | | | | | | | |
| | Hydroxypropyl | — | — | 5.0 | — | — | — | — |
| | methylcellulose | | | | | | | |
| | 6 cps | | | | | | | |
| | Ethyl cellulose | — | — | — | — | — | 5.0 | — |
| | 6-8 mPas | | | | | | | |
| | Polyethylene | 0.25 | 0.25 | 0.25 | 0.25 | 0.25 | 0.25 | — |
| | glycol 400 | | | | | | | |
| | Ethanol | — | — | — | 10.0 | 25.0 | 25.0 | — |
| | Purified | 50.0 | 50.0 | 50.0 | 40.0 | 25.0 | 25.0 | — |
| | water | | | | | | | |
| Enteric | Methacrylic acid | 50.0 | 50.0 | 50.0 | 50.0 | 50.0 | 50.0 | 50.0 |
| layer | copolymer | | | | | | | |
| | L30-D55 | | | | | | | |
| | Triethyl | 8.0 | 8.0 | 8.0 | 8.0 | 8.0 | 8.0 | 8.0 |
| | citrate | | | | | | | |
| | Polysorbate 80 | 0.2 | 0.2 | 0.2 | 0.2 | 0.2 | 0.2 | 0.2 |
| | Purified | 400.0 | 400.0 | 400.0 | 400.0 | 48.0 | 400.0 | 400.0 |
| | water | | | | | | | |

| | | | | | | | |
|---|---|---|---|---|---|---|---|
| Total weight | 607.95 | 607.95 | 607.95 | 607.95 | 607.95 | 607.95 | 602.7 |

(Unit: mg/tablet)

The total weight in the table above excludes the solvent weight.

### Test Example 2: Naproxen Dissolution Test

A naproxen dissolution test was performed in 900 mL of a pH 7.4 solution with respect to Examples 6 to 12 and Naprosyn EC 500 mg (Roche) according to the paddle method. The test solutions were collected at 5, 10, 15, 30, 45 and 60 minutes after starting the test, a dissolution rate of Naproxen was confirmed by using high performance liquid chromatography, and the results thereof are shown in table 4. The conditions for high performance liquid chromatography were the same as in above test example 1.

TABLE 4
Naproxen dissolution rate (%)

| Time | Example | Example | Example | Example | Example | Example | Example | Naprosyn |
|---|---|---|---|---|---|---|---|---|
| (min) | 6 | 7 | 8 | 9 | 10 | 11 | 12 | EC |
| 0 | 0.0 | 0.0 | 0.0 | 0.0 | 0.0 | 0.0 | 0.0 | 0.0 |
| 5 | 0.1 | 0.2 | 0.1 | 0.2 | 0.1 | 0.2 | 0.0 | 0.2 |
| 10 | 0.2 | 0.3 | 0.3 | 0.4 | 0.3 | 0.5 | 0.0 | 0.3 |
| 15 | 30.6 | 28.8 | 27.5 | 29.4 | 30.1 | 26.5 | 15.6 | 35.4 |
| 30 | 99.9 | 98.9 | 99.5 | 99.2 | 100.1 | 98.3 | 87.6 | 102.2 |
| 45 | 100.5 | 99.5 | 100.1 | 99.9 | 101.5 | 100.6 | 95.4 | 102.3 |
| 60 | 100.6 | 99.9 | 100.1 | 100.0 | 101.5 | 100.7 | 99.6 | 102.5 |

As shown in the results of Examples 6 to 10 and 11 in above table 4, it can be confirmed that there is no difference in the dissolution of naproxen according to the coating substrate of the primary separation layer (Examples 6 to 10: HPMC, Example 11: EC), HPMC viscosity and solvent, and solvent ratio. In addition, as shown in Example 12, when there is no primary separation layer, it can be confirmed that the dissolution of naproxen is delayed due to the direct influence of the enteric layer.

### Examples 13 to 19: Application of Enteric Layer for Each Enteric Coating Substrate

An enteric layer made of a methacrylic acid copolymer, triethyl citrate and polysorbate was coated and laminated on the tablet applied with the primary separation layer prepared as in Example 6. At this time, with the composition of table 5 below, coating was carried out for each substrate and ratio of methacrylic acid copolymer as in Examples 13 to 19.

TABLE 5

| | | Example | Example | Example | Example | Example | Example | Example |
|---|---|---|---|---|---|---|---|---|
| Classification | | 13 | 14 | 15 | 16 | 17 | 18 | 19 |
| Uncoated | Naproxen | 500.0 | 500.0 | 500.0 | 500.0 | 500.0 | 500.0 | 500.0 |
| core layer | Povidone | 10.0 | 10.0 | 10.0 | 10.0 | 10.0 | 10.0 | 10.0 |
| | Colloidal | 3.0 | 3.0 | 3.0 | 3.0 | 3.0 | 3.0 | 3.0 |
| | silicon | | | | | | | |
| | dioxide | | | | | | | |
| | Croscarmellose | 30.0 | 30.0 | 30.0 | 30.0 | 30.0 | 30.0 | 30.0 |
| | sodium | | | | | | | |
| | Magnesium | 1.5 | 1.5 | 1.5 | 1.5 | 1.5 | 1.5 | 1.5 |
| | stearate | | | | | | | |
| | Purified | 200.0 | 200.0 | 200.0 | 200.0 | 200.0 | 200.0 | 200.0 |
| | water | | | | | | | |
| Primary | Hydroxypropyl | 5.0 | 5.0 | 5.0 | 5.0 | 5.0 | 5.0 | 5.0 |
| separation | methylcellulose | | | | | | | |
| layer | 3 cps | | | | | | | |
| | Polyethylene | 0.25 | 0.25 | 0.25 | 0.25 | 0.25 | 0.25 | 0.25 |
| | glycol 400 | | | | | | | |
| | Purified | 50.0 | 50.0 | 50.0 | 50.0 | 50.0 | 40.0 | 25.0 |
| | water | | | | | | | |
| Enteric | Methacrylic acid | 50.0 | 45.0 | 40.0 | — | — | — | — |
| layer | copolymer | | | | | | | |
| | L30-D55 | | | | | | | |
| | Methacrylic acid | — | — | — | 50.0 | — | — | — |
| | copolymer | | | | | | | |
| | L100-D55 | | | | | | | |
| | Methacrylic acid | — | — | — | — | 50.0 | — | 25.0 |
| | copolymer | | | | | | | |
| | L100 | | | | | | | |
| | Methacrylic acid | — | — | — | — | — | 50.0 | 25.0 |
| | copolymer | | | | | | | |
| | S100 | | | | | | | |
| | Triethyl | 8.0 | 8.0 | 8.0 | 8.0 | 8.0 | 8.0 | 8.0 |
| | citrate | | | | | | | |
| | Polysorbate 80 | 0.2 | 0.2 | 0.2 | 0.2 | 0.2 | 0.2 | 0.2 |
| | 1N sodium | — | — | — | 0.5 | — | — | — |
| | hydroxide | | | | | | | |
| | Acetone | — | — | — | — | 380.0 | 380.0 | 380.0 |
| | Isopropyl | — | — | — | — | 570.0 | 570.0 | 570.0 |
| | alcohol | | | | | | | |
| | Purified | 400.0 | 400.0 | 400.0 | 400.0 | 48.0 | 48.0 | 48.0 |
| | water | | | | | | | |

| | | | | | | | |
|---|---|---|---|---|---|---|---|
| Total weight | 607.95 | 602.95 | 597.95 | 608.45 | 607.95 | 607.95 | 607.95 |

(Unit: mg/tablet)

The total weight in the table above excludes the solvent weight.

### Test Example 3: Acid Resistance Test

An acid resistance test was carried out for the prepared Examples 13 to 19. An acid resistance evaluation was performed based on the dissolution rate of naproxen being less than 10% by evaluating the dissolution rate of naproxen for two hours in a dissolution device with 0.1N HCl solution according to the Korean Pharmacopoeia. The conditions for high performance liquid chromatography were the same as the conditions for above test example 1. The results of acid resistance evaluation are as shown in table 6 below.

TABLE 6
Naproxen dissolution rate (%)

| Time | Example | Example | Example | Example | Example | Example | Example | Naprosyn |
|---|---|---|---|---|---|---|---|---|
| (min) | 13 | 14 | 15 | 16 | 17 | 18 | 19 | EC |
| 0 | 0.0 | 0.0 | 0.0 | 0.0 | 0.0 | 0.0 | 0.0 | 0.0 |
| 30 | 0.0 | 0.0 | 1.1 | 0.0 | 0.0 | 0.0 | 0.0 | 0.0 |
| 60 | 0.0 | 0.5 | 2.6 | 0.0 | 0.0 | 0.0 | 0.0 | 0.0 |
| 90 | 0.0 | 1.2 | 4.3 | 0.0 | 0.0 | 0.0 | 0.0 | 0.0 |
| 120 | 0.0 | 3.7 | 7.9 | 0.0 | 0.0 | 0.0 | 0.0 | 0.0 |

As can be understood from above table 6, it can be confirmed that there is a difference in acid resistance according to the content of the enteric coated layer as shown in the results of Examples 13 to 15. In addition, as shown in the results of Examples 16 to 19, it can be confirmed that there is no difference in acid resistance according to the type and solvent of the enteric coating substrate.

### Examples 20 to 28: Application of Secondary Separation Layer and Tegoprazan Layer

A secondary separation layer was coated and laminated on the surface of the tablet including the enteric layer of Example 12 or the surface of the tablet including the primary separation layer and the enteric layer of Example 13 by using the solution of hydroxypropylmethylcellulose or ethylcellulose prepared as shown in the composition of table 7 below. As an active ingredient, tegoprazan was coated and laminated thereon by using a solution made of tegoprazan, hydroxypropylmethylcellulose, polyethylene glycol, and polysorbate as shown in the composition of table 7 below (Examples 20 to 24, 27 and 28). In Examples 25 and 26, a tegoprazan coated layer was directly coated and laminated without coating the secondary separation layer as shown in table 7 below.

TABLE 7

| | | Example | Example | Example | Example | Example | Example | Example | Example | Example |
|---|---|---|---|---|---|---|---|---|---|---|
| Classification | | 20 | 21 | 22 | 23 | 24 | 25 | 26 | 27 | 28 |
| Uncoated | Naproxen | 500.0 | 500.0 | 500.0 | 500.0 | 500.0 | 500.0 | 500.0 | 500.0 | 500.0 |
| core layer | Povidone | 10.0 | 10.0 | 10.0 | 10.0 | 10.0 | 10.0 | 10.0 | 10.0 | 10.0 |
| | Colloidal | 3.0 | 3.0 | 3.0 | 3.0 | 3.0 | 3.0 | 3.0 | 3.0 | 3.0 |
| | silicon | | | | | | | | | |
| | dioxide | | | | | | | | | |
| | Croscarmellose | 30.0 | 30.0 | 30.0 | 30.0 | 30.0 | 30.0 | 30.0 | 30.0 | 30.0 |
| | sodium | | | | | | | | | |
| | Magnesium | 1.5 | 1.5 | 1.5 | 1.5 | 1.5 | 1.5 | 1.5 | 1.5 | 1.5 |
| | stearate | | | | | | | | | |
| | Purified | 200.0 | 200.0 | 200.0 | 200.0 | 200.0 | 200.0 | 200.0 | 200.0 | 200.0 |
| | water | | | | | | | | | |
| Primary | Hydroxypropyl | 5.0 | 5.0 | 5.0 | 5.0 | — | 5.0 | — | 5.0 | 5.0 |
| separation | methylcellulose | | | | | | | | | |
| layer | 3 cps | | | | | | | | | |
| | Polyethylene | 0.25 | 0.25 | 0.25 | 0.25 | — | 0.25 | — | 0.25 | 0.25 |
| | glycol 400 | | | | | | | | | |
| | Purified | 50.0 | 50.0 | 50.0 | 50.0 | — | 50.0 | — | 50.0 | 50.0 |
| | water | | | | | | | | | |
| Enteric | Methacrylic acid | 50.0 | 50.0 | 50.0 | 50.0 | 50.0 | 50.0 | 50.0 | 50.0 | 50.0 |
| layer | copolymer | | | | | | | | | |
| | L30-D55 | | | | | | | | | |
| | Triethyl | 8.0 | 8.0 | 8.0 | 8.0 | 8.0 | 8.0 | 8.0 | 8.0 | 8.0 |
| | citrate | | | | | | | | | |
| | Polysorbate 80 | 0.2 | 0.2 | 0.2 | 0.2 | 0.2 | 0.2 | 0.2 | 0.2 | 0.2 |
| | Purified | 400.0 | 400.0 | 400.0 | 400.0 | 400.0 | 400.0 | 400.0 | 400.0 | 400.0 |
| | water | | | | | | | | | |
| Secondary | Hydroxypropyl | 20.0 | — | — | — | 20.0 | — | — | 20.0 | 20.0 |
| separation | methylcellulose | | | | | | | | | |
| layer | 3 cps | | | | | | | | | |
| | Hydroxypropyl | — | 20.0 | — | — | — | — | — | — | — |
| | methylcellulose | | | | | | | | | |
| | 4.5 cps | | | | | | | | | |
| | Hydroxypropyl | — | — | 20.0 | — | — | — | — | — | — |
| | methylcellulose | | | | | | | | | |
| | 6 cps | | | | | | | | | |
| | Ethyl cellulose | — | — | — | 20.0 | — | — | — | — | — |
| | 6-8 mPas | | | | | | | | | |
| | Polyethylene | 0.9 | 0.9 | 0.9 | 0.9 | 0.9 | — | — | 0.9 | 0.9 |
| | glycol 400 | | | | | | | | | |
| | Ethanol | — | — | — | 100.0 | — | — | — | — | — |
| | Purified | 200.0 | 200.0 | 200.0 | 100.0 | — | — | — | 200.0 | 200.0 |
| | water | | | | | | | | | |
| Tegoprazan | Tegoprazan | 25.0 | 25.0 | 25.0 | 25.0 | 25.0 | 25.0 | 25.0 | 25.0 | 25.0 |
| coated | crystalline | | | | | | | | | |
| layer | Hydroxypropyl | 38.0 | 38.0 | 38.0 | 38.0 | 38.0 | 38.0 | 38.0 | — | — |
| | methylcellulose | | | | | | | | | |
| | 3 cps | | | | | | | | | |
| | Hydroxypropyl | — | — | — | — | — | — | — | 38.0 | — |
| | methylcellulose | | | | | | | | | |
| | 4.5 cps | | | | | | | | | |
| | Hydroxypropyl | — | — | — | — | — | — | — | — | 38.0 |
| | methylcellulose | | | | | | | | | |
| | 6 cps | | | | | | | | | |
| | Polyethylene | 2.0 | 2.0 | 2.0 | 2.0 | 2.0 | 2.0 | 2.0 | 2.0 | 2.0 |
| | glycol 400 | | | | | | | | | |
| | Polysorbate 80 | 4.0 | 4.0 | 4.0 | 4.0 | 4.0 | 4.0 | 4.0 | 4.0 | 4.0 |
| | Purified | 506.0 | 506.0 | 506.0 | 506.0 | 506.0 | 506.0 | 506.0 | 506.0 | 506.0 |
| | water | | | | | | | | | |

| | | | | | | | | | |
|---|---|---|---|---|---|---|---|---|---|
| Total weight | 697.85 | 697.85 | 697.85 | 697.85 | 692.6 | 676.95 | 671.7 | 697.85 | 697.85 |

(Unit: mg/tablet)

The total weight in the table above excludes the solvent weight.

As shown in above table 7, the tablets of Examples 20 to 23, 27 and 28 include four coated layers of a primary separation layer-enteric layer-secondary separation layer-tegoprazan coated layer on a naproxen core tablet. Example 24 includes three coated layers of an enteric layer-secondary separation layer-tegoprazan coated layer on a naproxen core tablet. Example 25 includes three coated layers of a primary separation layer-enteric layer-tegoprazan coated layer on a naproxen core tablet. Example 26 includes two coated layers of an enteric layer-tegoprazan coated layer on a naproxen core tablet.

### Test Example 4: Results of Tegoprazan Dissolution Test

A tegoprazan dissolution test was performed in 900 mL of a pH 1.2 solution with respect to prepared Examples 20 to 28 according to the paddle method in the Korean Pharmacopoeia. The test solutions were collected at 5, 10, 15, 30, 45 and 60 minutes after starting the test, a dissolution rate of tegoprazan was confirmed by using high performance liquid chromatography, and the specific test conditions are as follows.

### <High Performance Liquid Chromatography Conditions>

Column: A column filled with 5 μm of octadecylsilyl silica gel for liquid chromatography in a stainless steel tube with an inner diameter of about 4.6 mm and a length of about 15 cm or a column equivalent thereto
Column temperature: Constant temperature around 30° C.
Detector: Ultraviolet absorption spectrophotometer (wavelength: 262 nm)
Flux: 1.0 mL/min
Mobile phase: Mixture of 0.01 mol/L ammonium acetate buffer: Acetonitrile (11:9)
(Prepared by dissolving 0.01 mol/L ammonium acetate buffer: ammonium acetate (CH3COONH4) 0.77 g in 1 L of water and filtering with a 0.45 μm membrane filter)
Eluate: pH 1.2 solution, 900 mL, paddle method, 50 rpm

The results of tegoprazan dissolution test of Examples 20 to 28 are shown in table 8.

TABLE 8
Tegoprazan dissolution rate (%)

| Time | Example | Example | Example | Example | Example | Example | Example | Example | Example |
|---|---|---|---|---|---|---|---|---|---|
| (min) | 20 | 21 | 22 | 23 | 24 | 25 | 26 | 27 | 28 |
| 0 | 0.0 | 0.0 | 0.0 | 0.0 | 0.0 | 0.0 | 0.0 | 0.0 | 0.0 |
| 5 | 28.6 | 27.1 | 26.5 | 27.5 | 25.3 | 24.9 | 29.8 | 29.3 | 26.8 |
| 10 | 52.9 | 50.6 | 49.2 | 51.0 | 50.6 | 48.6 | 53.6 | 54.1 | 51.7 |
| 15 | 66.3 | 64.1 | 62.9 | 65.1 | 61.8 | 62.0 | 68.1 | 68.5 | 65.2 |
| 30 | 80.2 | 77.6 | 76.5 | 79.9 | 76.1 | 75.6 | 82.6 | 81.9 | 78.4 |
| 45 | 85.8 | 82.3 | 80.8 | 83.4 | 81.6 | 80.1 | 89.4 | 87.5 | 83.2 |
| 60 | 96.9 | 92.7 | 91.9 | 95.2 | 93.8 | 91.6 | 97.6 | 97.0 | 95.0 |

As can be understood from above table 8, it can be confirmed that there is no difference in tegoprazan dissolution according to each the viscosity and type of the secondary separation layer substrate as shown in the results of Examples 20 to 23. In addition, from the results of Example 20 and Examples 24 to 26, it can be confirmed that there is no difference in the dissolution rate of tegoprazan according to the presence or absence of the primary separation layer and/or the secondary separation layer. In addition, from the results of Example 20 and Examples 27 to 28, it can be confirmed that there is no difference in the dissolution rate of tegoprazan according to the viscosity of the coating substrate (HPMC) of the tegoprazan coated layer.

### Test Example 5: Results of Tegoprazan Stability Test

The prepared Examples 20 to 28 were put into an HDPE bottle and stored under stress conditions (60° C., 80% RH) for four weeks, after which an increase in the amount of impurities was confirmed by using high-performance liquid chromatography (initial and after four weeks of storage under stress conditions (stress week 4). The specific test conditions are as follows:

### <High Performance Liquid Chromatography Conditions>

Column: A column filled with 2.7 μm of octadecylsilyl silica gel for liquid chromatography in a stainless steel tube with an inner diameter of about 4.6 mm and a length of about 15 cm
Column temperature: Constant temperature around 30° C.
Detector: Ultraviolet absorption spectrophotometer (wavelength: 220 nm)
Flux: 0.8 mL/min
Measurement range: For 25 minutes after test liquid infusion
Mobile phase A: Mixture of 0.01 mol/L ammonium acetate buffer: acetonitrile (19:1)
(Prepared by dissolving 0.01 mol/L ammonium acetate buffer: ammonium acetate (CH3COONH4) 0.77 g in water to make 1 L, and adjusting the pH to 6.5±0.2 with dilute acetic acid and filtering through a 0.45 μm membrane filter)
Mobile phase B: Acetonitrile
Mobile phase gradient conditions

TABLE 9

| Time (min) | Mobile phase A (vol %) | Mobile phase B (vol %) |
|---|---|---|
| 0~2 | 88 | 12 |
| 2~3 | 88 → 70 | 12 → 30 |
| 3~15 | 70 | 30 |
| 15~16 | 70 → 20 | 30 → 80 |
| 16~17 | 20 → 10 | 80 → 90 |
| 17~20 | 10 | 90 |
| 20~20.01 | 10 → 88 | 90 → 12 |
| 20.01~25 | 88 | 12 |

The results of tegoprazan stability test are shown in table 10.

TABLE 10

| Total | Example | Example | Example | Example | Example | Example | Example | Example | Example |
|---|---|---|---|---|---|---|---|---|---|
| impurities | 20 | 21 | 22 | 23 | 24 | 25 | 26 | 27 | 28 |
| Initial | 0.07 | 0.06 | 0.06 | 0.07 | 0.07 | 0.06 | 0.07 | 0.08 | 0.07 |
| Stress | 0.11 | 0.18 | 0.12 | 0.13 | 0.14 | 0.22 | 0.24 | 0.12 | 0.15 |
| week 4 | | | | | | | | | |

(Criteria for total impurities: 1.0% or less)

According to the results of above table 10, it can be confirmed that there is no difference in the stability of tegoprazan according to the presence or absence of the secondary separation layer and the configuration of the secondary separation layer. In addition, it can be confirmed that all of Examples 20 to 28 are stable with a value of 1.0% or less, which is a reference value of total impurities.

### Test Example 6: Results of Stability Test of Tegoprazan Coating Solution

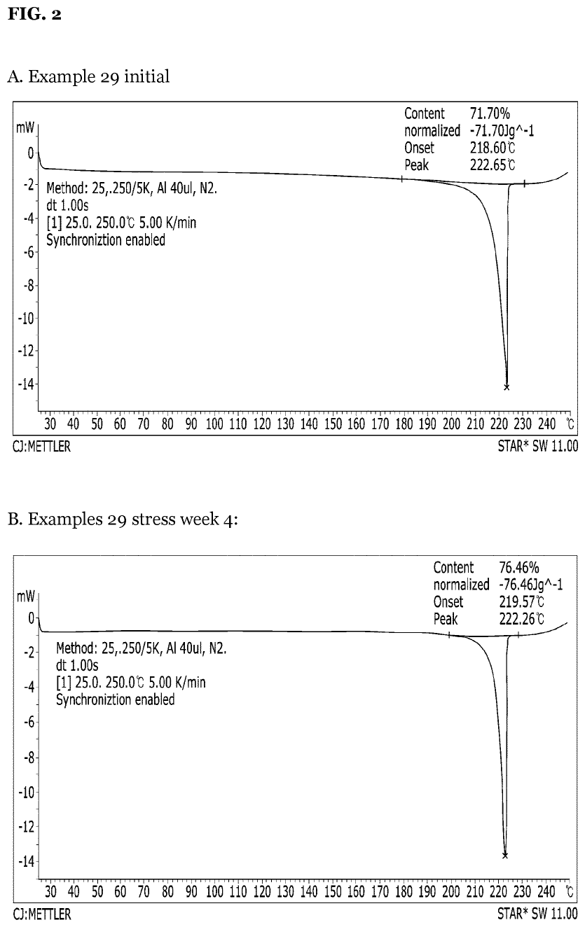

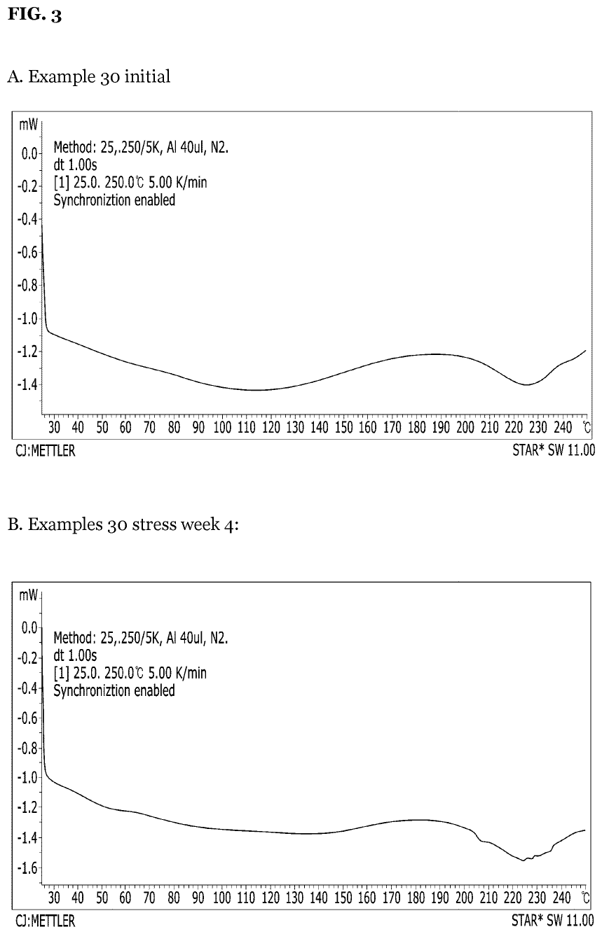

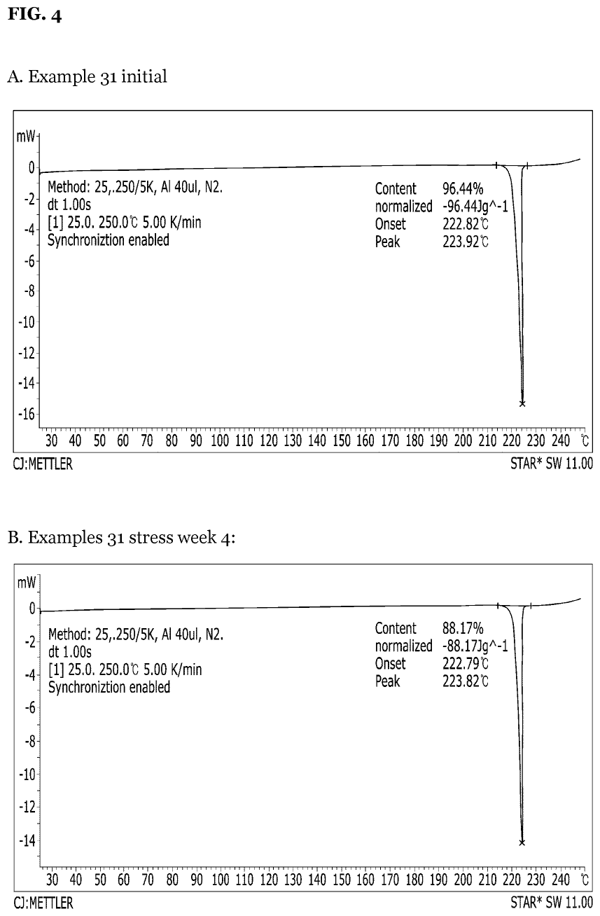

As shown in the composition of table 11 below, a tegoprazan, which is an active ingredient, coating solution was prepared from a solution made of tegoprazan, hydroxypropyl methylcellulose, polyethylene glycol, and polysorbate. Specifically, as shown in the composition of table 11 below, a coating solution containing tegoprazan crystalline, amorphous and polymorphic crystal forms was prepared, put into a 20 mL glass vial, and stored under stress conditions (60° C., 80% RH) for four weeks, so as to observe a change in the crystal form through DSC. The results thereof were shown in FIGS. 2 to 4.

TABLE 11

| | | Example | Example | Example | Example | Example |
|---|---|---|---|---|---|---|
| Classification | | 29 | 30 | 31 | 32 | 33 |
| Tegoprazan | Tegoprazan | 25.00 | — | — | — | — |
| coating | crystalline | | | | | |
| solution | Tegoprazan | — | 25.00 | — | — | — |
| | amorphous | | | | | |
| | Tegoprazan | — | — | 25.00 | — | — |
| | pidolate salt | | | | | |
| | Tegoprazan | — | — | — | 25.00 | — |
| | citrate salt | | | | | |
| | Tegoprazan | — | — | — | — | 25.00 |
| | malate salt | | | | | |
| | HPMC 3 cps | 38.00 | 38.00 | 38.00 | 38.00 | 38.00 |
| | Polyethylene | 2.00 | 2.00 | 2.00 | 2.00 | 2.00 |
| | glycol 400 | | | | | |
| | Polysorbate 80 | 4.00 | 4.00 | 4.00 | 4.00 | 4.00 |
| | Purified | 506.00 | 506.00 | 506.00 | 506.00 | 506.00 |
| | water | | | | | |

| | | | | | |
|---|---|---|---|---|---|
| Total weight | 575.0 | 575.0 | 575.0 | 575.0 | 575.0 |

(Unit: mg/tablet)

As can be understood from FIGS. 2 to 4, it can be confirmed that no change in crystalline form is observed from the composition of the coating solution made of tegoprazan crystalline, amorphous and polymorphic crystal forms.

### Examples 34 to 39: Application of Tegoprazan Coated Layer According to Tegoprazan Crystal Form and Colored Layer

As shown in the composition of table 12 below, tegoprazan, which is an active ingredient, was coated and laminated by using a solution made of tegoprazan, hydroxypropyl methylcellulose, polyethylene glycol, and polysorbate, so as to prepare a coated tablet with a fourth layer. A fifth colored layer was coated and laminated thereon by using a solution made of opadry white and purified water so as to prepare multi-coated tablets of Examples 34 to 39.

TABLE 12

| | | Example | Example | Example | Example | Example | Example |
|---|---|---|---|---|---|---|---|
| Classification | | 34 | 35 | 36 | 37 | 38 | 39 |
| Uncoated | Naproxen | 500.0 | 500.0 | 500.0 | 500.0 | 500.0 | 500.0 |
| core layer | Povidone | 10.0 | 10.0 | 10.0 | 10.0 | 10.0 | 10.0 |
| | Colloidal | 3.0 | 3.0 | 3.0 | 3.0 | 3.0 | 3.0 |
| | silicon | | | | | | |
| | dioxide | | | | | | |
| | Croscarmellose | 30.0 | 30.0 | 30.0 | 30.0 | 30.0 | 30.0 |
| | sodium | | | | | | |
| | Magnesium | 1.5 | 1.5 | 1.5 | 1.5 | 1.5 | 1.5 |
| | stearate | | | | | | |
| | Purified | 200.0 | 200.0 | 200.0 | 200.0 | 200.0 | 200.0 |
| | water | | | | | | |
| Primary | Hydroxypropyl | 5.0 | 5.0 | 5.0 | 5.0 | 5.0 | 5.0 |
| separation | methylcellulose | | | | | | |
| layer | 3 cps | | | | | | |
| | Polyethylene | 0.25 | 0.25 | 0.25 | 0.25 | 0.25 | 0.25 |
| | glycol 400 | | | | | | |
| | Purified | 50.0 | 50.0 | 50.0 | 50.0 | 50.0 | 50.0 |
| | water | | | | | | |
| Enteric | Methacrylic acid | 50.0 | 50.0 | 50.0 | 50.0 | 50.0 | — |
| layer | copolymer | | | | | | |
| | L30-D55 | | | | | | |
| | Methacrylic acid | — | — | — | — | — | 25.0 |
| | copolymer | | | | | | |
| | L100 | | | | | | |
| | Methacrylic acid | — | — | — | — | — | 25.0 |
| | copolymer | | | | | | |
| | S100 | | | | | | |
| | Triethyl | 8.0 | 8.0 | 8.0 | 8.0 | 8.0 | 8.0 |
| | citrate | | | | | | |
| | Polysorbate 80 | 0.2 | 0.2 | 0.2 | 0.2 | 0.2 | 0.2 |
| | Acetone | — | — | — | — | — | 380.0 |
| | Isopropyl | — | — | — | — | — | 570.0 |
| | alcohol | | | | | | |
| | Purified | 400.0 | 400.0 | 400.0 | 400.0 | 400.0 | 48.0 |
| | water | | | | | | |
| Secondary | Hydroxypropyl | 20.0 | 20.0 | 20.0 | 20.0 | 20.0 | 20.0 |
| separation | methylcellulose | | | | | | |
| layer | 3 cps | | | | | | |
| | Polyethylene | 0.9 | 0.9 | 0.9 | 0.9 | 0.9 | 0.9 |
| | glycol 400 | | | | | | |
| | Purified | 188.0 | 188.0 | 188.0 | 188.0 | 188.0 | 188.0 |
| | water | | | | | | |
| Tegoprazan | Tegoprazan | 25.00 | — | — | — | — | 25.00 |
| coated | crystalline | | | | | | |
| layer | Tegoprazan | — | 25.00 | — | — | — | — |
| | amorphous | | | | | | |
| | Tegoprazan | — | — | 25.00 | — | — | — |
| | pidolate salt | | | | | | |
| | Tegoprazan | — | — | — | 25.00 | — | — |
| | citrate salt | | | | | | |
| | Tegoprazan | — | — | — | — | 25.00 | — |
| | malate salt | | | | | | |
| | HPMC 3 cps | 38.00 | 38.00 | 38.00 | 38.00 | 38.00 | 38.00 |
| | Polyethylene | 2.00 | 2.00 | 2.00 | 2.00 | 2.00 | 2.00 |
| | glycol 400 | | | | | | |
| | Polysorbate 80 | 4.00 | 4.00 | 4.00 | 4.00 | 4.00 | 4.00 |
| | Purified | 506.00 | 506.00 | 506.00 | 506.00 | 506.00 | 506.00 |
| | water | | | | | | |
| Colored | Opadry | 10.55 | 10.55 | 10.55 | 10.55 | 10.55 | 10.55 |
| coated | white | | | | | | |
| layer | Purified | 95.0 | 95.0 | 95.0 | 95.0 | 95.0 | 95.0 |
| | water | | | | | | |

| | | | | | | |
|---|---|---|---|---|---|---|
| Total weight | 708.4 | 708.4 | 708.4 | 708.4 | 708.4 | 708.4 |

(Unit: mg/tablet)

The total weight in the table above excludes the solvent weight.

### Test Example 7: Tegoprazan Stability Test

The prepared Examples 34 to 39 were put into an HDPE bottle and stored under stress conditions (60° C., 80% RH) for four weeks, after which an increase in the amount of impurities was confirmed by using high-performance liquid chromatography. The conditions for high performance liquid chromatography were the same as the conditions for above test example 5. The results thereof are shown in table 13.

TABLE 13

| Total | Example | Example | Example | Example | Example | Example |
|---|---|---|---|---|---|---|
| impurities | 34 | 35 | 36 | 37 | 38 | 39 |
| Initial | 0.07 | 0.06 | 0.05 | 0.05 | 0.06 | 0.07 |
| Stress | 0.11 | 0.15 | 0.12 | 0.17 | 0.16 | 0.18 |
| week 4 | | | | | | |

(Criteria for total impurities: 1.0% or less)

As understood from the results of above table 13, it can be confirmed that there is no difference in the stability of tegoprazan according to tegoprazan amorphous and crystalline forms and tegoprazan salts. In particular, it was confirmed that all of Examples 34 to 39 exhibit results of 1.0% or less, which is a criterion for total impurities. In other words, it can be confirmed that the pharmaceutical composition of the present invention has excellent stability of tegoprazan.

### Test Example 8: Tegoprazan Dissolution Test

A tegoprazan dissolution test was performed in 900 mL of a pH 1.2 solution with respect to prepared Examples 34 to 39 according to the paddle method in the Korean Pharmacopoeia. The test solutions were collected at 5, 10, 15, 30, 45 and 60 minutes after starting the test, a dissolution rate of tegoprazan was confirmed by using high performance liquid chromatography, and the conditions for high performance liquid chromatography were the same as the conditions for above test example 4. The results thereof are shown in table 14.

TABLE 14
Tegoprazan dissolution rate (%)

| Time | Example | Example | Example | Example | Example | Example |
|---|---|---|---|---|---|---|
| (min) | 34 | 35 | 36 | 37 | 38 | 39 |
| 0 | 0.0 | 0.0 | 0.0 | 0.0 | 0.0 | 0.0 |
| 5 | 27.5 | 29.1 | 25.3 | 30.0 | 28.1 | 26.9 |
| 10 | 52.6 | 55.6 | 49.9 | 57.3 | 52.8 | 50.9 |
| 15 | 69.9 | 70.2 | 68.2 | 74.4 | 72.3 | 68.5 |
| 30 | 82.7 | 82.6 | 79.4 | 83.1 | 82.1 | 80.6 |
| 45 | 87.6 | 88.9 | 83.4 | 89.9 | 87.9 | 85.4 |
| 60 | 97.0 | 97.2 | 95.1 | 98.3 | 97.6 | 94.6 |

As shown in the results of above table 14, it can be confirmed that there is no difference in the dissolution rate of tegoprazan according to tegoprazan crystalline and amorphous forms and tegoprazan salts.

### Test Example 9: Acid Resistance Test

An acid resistance test was carried out for prepared Example 34. An acid resistance evaluation was performed based on the dissolution rate of naproxen being less than 10% by evaluating the dissolution rate of naproxen for two hours in a dissolution device with 0.1N HCl solution and pH 4.0 solution according to the Korean Pharmacopoeia. The conditions for high performance liquid chromatography were the same as the conditions for above test example 1. The results of acid resistance evaluation are as shown in table 15 below.

TABLE 15
Example 34 Naproxen dissolution rate (%)

| Time (min) | 0.1N HCl | pH 4.0 |
|---|---|---|
| 0 | 0.0 | 0.0 |
| 30 | 0.0 | 0.0 |
| 60 | 0.0 | 0.0 |
| 90 | 0.0 | 0.0 |
| 120 | 0.0 | 0.0 |

As understood from in above table 15, it can be confirmed that naproxen is not eluted for two hours in 0.1N HCl solution and pH 4.0 solution with respect to Example 34. Accordingly, it can be understood that the pharmaceutical composition of the present invention will not release naproxen in the gastric juice environment.

### Test Example 10: In-Vivo Pharmacokinetic Parameter Test

The bioavailability of naproxen and tegoprazan was confirmed with minipigs by using the prepared Example 34. A randomized crossover study of eight minipigs was conducted and K-CAB tablet 25 mg and Naprosyn EC 500 mg were co-administered as control drugs.

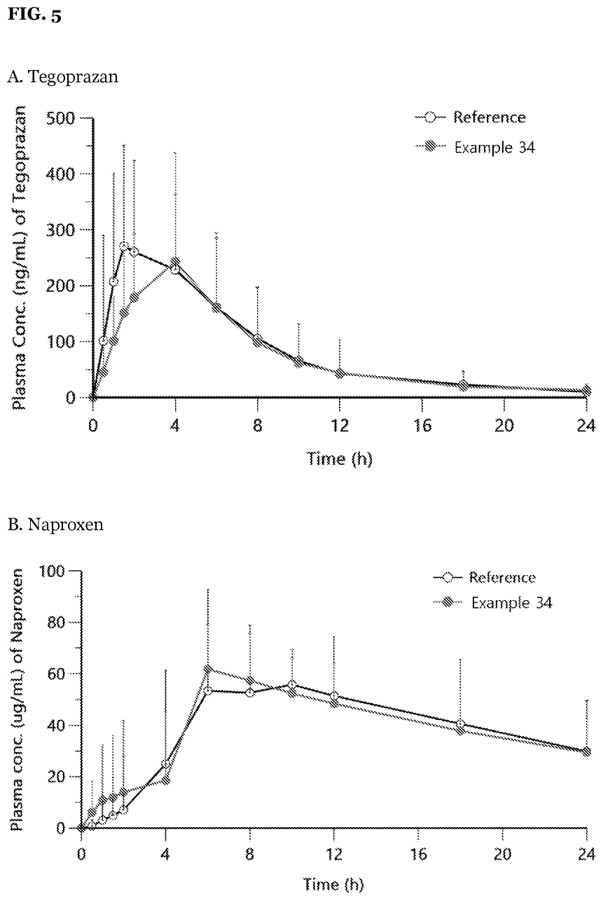

Specifically, an animal was set by two assistants not to move at all, after which a test substance was inserted into the inside of the tongue, and then the mouth was closed and the pharyngolarynx was gently stroked to allow deglutition, so as to confirm if the animal swallows up. Then, the animal is fed with about 10 mL of water by using a syringe. The animal was fasted without feed for 16 hours or more (over-night fasting) before administration, and feed was administered 4 hours after administration of the test substance. Blood was collected once before administration and collected again at 0.5, 1, 1.5, 2, 4, 6, 8, 10, 12, 18, and 24 hours (a total of 12 time points) after administration of test drug or control drug. About 3.0 mL of blood sample was collected from the cephalic vein at each blood sampling time. Pharmacokinetic parameter results are shown in FIG. 5.

As shown in the results of FIG. 5, it can be confirmed that the pharmacokinetic parameters of naproxen and tegoprazan of Example 34 exhibit sufficient pK parameters as in the co-administration of the control drug. Thus, it can be understood that Example 34 shows an excellent therapeutic effect as in the co-administration of the control drug.

Accordingly, the pharmaceutical composition of the present invention is a combination preparation formulated with tegoprazan and non-steroidal anti-inflammatory drug in one unit dosage form, has excellent convenience of and compliance to medication, and shows pharmacokinetic parameters such as bioavailability, etc., similar to those when co-administered with a commercially available control drug, thereby exhibiting an excellent effect without reducing the medicinal efficacy of each ingredient.

1. A pharmaceutical composition comprising:
a first compartment containing a first active ingredient; and a second compartment containing a second active ingredient,
wherein the first active ingredient is formulated for controlled release, and the second active ingredient is formulated for immediate release,
in which the first active ingredient is a non-steroidal anti-inflammatory drug (NSAID) in the amount of about 250 mg to about 1000 mg, and
the second active ingredient is tegoprazan, an optical isomer thereof, a pharmaceutically acceptable salt thereof, a hydrate or solvate thereof, or a mixture thereof, in the amount of about 20 mg to about 100 mg.

2. The pharmaceutical composition of claim 1, wherein the tegoprazan or the pharmaceutically acceptable salt thereof is in an amorphous or crystalline form.

3. The pharmaceutical composition of claim 1, wherein the pharmaceutical composition is a unit dosage form of a combination preparation comprising both the first active ingredient and the second active ingredient.

4. The pharmaceutical composition of claim 3, wherein the unit dosage form comprises a first compartment containing the first active ingredient, and a second compartment containing the second active ingredient,
in which the first compartment containing the first active ingredient includes a particle containing:
a core having the first active ingredient; and
an enteric coated layer located on the core and surrounding the core.

5. The pharmaceutical composition of claim 4, wherein the particle is a tablet, pellet or granule.

6. The pharmaceutical composition of claim 4, wherein the unit dosage form is a tablet.

7. The pharmaceutical composition of claim 4, wherein the second compartment comprising the second active ingredient is a second active ingredient layer containing the second active ingredient.

8. The pharmaceutical composition of claim 7, wherein the second active ingredient layer is a coated layer located on and surrounding the first compartment comprising the first active ingredient.

9. The pharmaceutical composition of claim 7, wherein the second active ingredient layer is laminated on the first compartment comprising the first active ingredient.

10. The pharmaceutical composition of claim 9, wherein the second active ingredient layer comprises a particle containing the second active ingredient.

11. The pharmaceutical composition of claim 10, wherein the particle comprising the second active ingredient is a tablet, pellet or granule.

12. The pharmaceutical composition of claim 7, wherein a separation layer for blocking a contact between the first active ingredient and the second active ingredient is f: between the core and the enteric coated layer of the first compartment or is between the first compartment and the second compartment.

13. The pharmaceutical composition of claim 4, wherein the unit dosage form is a capsule.

14. The pharmaceutical composition of claim 13, wherein the first compartment comprising the first active ingredient is a particle comprising:
a core containing the first active ingredient; and
an enteric coated layer located on the core and surrounding the core, and
wherein the second compartment comprising the second active ingredient is a particle comprising the second active ingredient.

15. The pharmaceutical composition of claim 14, wherein the capsule is filled with the particle comprising the first active ingredient and the particle comprising the second active ingredient.

16. The pharmaceutical composition of claim 15, wherein the particle comprising the first active ingredient and the particle comprising the second active ingredient are each independently a tablet, pellet, granule, or mixture thereof.

17. The pharmaceutical composition of claim 13, wherein the capsule is filled with a tablet,
wherein the tablet comprises the first compartment containing the first active ingredient; and the second compartment containing the second active ingredient, in which the first compartment containing the first active ingredient includes a particle containing:
a core having the first active ingredient; and
an enteric coated layer located on the core and surrounding the core, and
the second compartment containing the second active ingredient includes a particle containing the second active ingredient or a second active ingredient layer containing the second active ingredient.

18. The pharmaceutical composition of claim 4, wherein the enteric coated layer is about 5 wt % or more and about 20 wt % or less based on the total weight of the core.

19. The pharmaceutical composition of claim 1, wherein the non-steroidal anti-inflammatory drug comprises naproxen or a pharmaceutically acceptable salt thereof.

20. (canceled)

21. (canceled)

22. (canceled)

23. (canceled)

24. (canceled)

25. (canceled)
